(19) United States
(12) Patent Application Publication
(10) Pub. No.: US 2024/0016786 A1
(43) Pub. Date: Jan. 18, 2024

(54) GLUCOSIDASE INHIBITORS FOR THE TREATMENT AND PREVENTION OF PULMONARY INFECTIONS

(71) Applicant: COVIRIX Medical Pty Ltd, Bentleigh East (AU)

(72) Inventors: Sebastian Mario MARCUCCIO, Scoresby (AU); David Alexander Vodden MORTON, Altona (AU); Simon Peter TUCKER, Lysterfield (AU)

(21) Appl. No.: 18/022,840

(22) PCT Filed: Aug. 25, 2021
(86) PCT No.: PCT/AU2021/050978

§ 371 Date: Feb. 23, 2023

(30) Foreign Application Priority Data
Aug. 26, 2020 (AU) 2020903051

(51) Int. Cl. A61K 31/44; A61P 31/14; A61K 9/00; A61K 31/437

(52) U.S. Cl. CPC A61K 31/44; A61P 31/14; A61K 9/0075; A61K 31/437

(57) ABSTRACT

The present invention relates generally to glucosidase inhibitors and their use in treating or preventing pulmonary diseases. In particular, the present invention is directed to use of α-glucosidase inhibitors for treating or preventing bacterial or viral infections of the respiratory tract, including SARS-CoV-2.

### RELATED APPLICATIONS

This application claims priority from Australian Provisional Patent Application No. 2020903051, the entire contents of which are incorporated herein by cross-reference.

### TECHNICAL FIELD

The present invention relates generally to glucosidase inhibitors and their use in treating or preventing pulmonary diseases. In particular, the present invention is directed to use of α-glucosidase inhibitors for treating or preventing bacterial or viral infections of the respiratory tract.

### BACKGROUND

Respiratory tract infections (RTIs) affect millions of people and are responsible for hundreds of thousands of deaths worldwide each year. RTIs can affect the upper or lower respiratory tract, including the sinuses, throat, airways or lungs, and may be bacterial or viral in nature. Upper RTIs include the common cold, laryngitis, pharyngitis, acute rhinitis and acute rhinosinusitis. Lower RTIs include acute bronchitis, bronchiolitis, pneumonia and tracheitis. Symptoms of RTIs can range from mild to severe, with severe cases often requiring hospitalisation and ventilation.

The novel severe acquired respiratory syndrome coronavirus 2 (SARS-CoV-2), the strain of coronavirus responsible for the COVID-19 pandemic, is an RTI that has infected more than 200 million people worldwide and caused nearly 4.4 million deaths. Accordingly, there is an immediate need for treatment options for SARS-CoV-2. The most expeditious strategy for developing treatments for SARS-CoV-2, among other RTIs, involves the repurposing of known therapeutic agents. There are a number of such agents currently in various stages of clinical development for the treatment of SARS-CoV-2, including remdesivir and favipiravir, both of which selectively inhibit the viral RNA-dependent RNA polymerase.

Entry of SARS-CoV-2 into host cells in both the upper and lower respiratory tract is mediated by heavily glycosylated trimeric spike (S) glycoproteins projecting from the surface of the virus. The Spike glycoproteins bind to the angiotensin-converting enzyme 2 (ACE2) receptor, leading to fusion of the virus with the host cell membrane. The fusion process involves large conformational changes of the Spike protein. Once inside the cell, viral polyproteins are synthesized that encode for the replication machinery required to synthesize new RNA via its RNA-dependent RNA polymerase activity. Structural proteins are then synthesized leading to completion of assembly and release of viral particles. This includes the Spike glycoprotein, which is a transmembrane protein and undergoes folding and glycosylation through the endoplasmic reticulum (ER) before being secreted and exposed on the plasma membrane.

A recent study by Rajasekharan et al. (2020) demonstrated that miglustat (N-butyl-1-deoxynojirimycin; NB-DNJ), an α-glucosidase inhibitor (AGI), which is currently marketed as Zavesca™ for the treatment of rare genetic lysosome storage diseases such as Gaucher and Niemann-Pick type C, has in vitro activity against SARS-CoV-2. The mechanism of action was demonstrated to reside in the inhibitory activity of miglustat towards α-glucosidases I and II present in the ER, which sequentially trim the three terminal glucose moieties on the N-linked glycans attached to the Spike glycoproteins. These reactions are essential for proper folding and function of Spike (among other glycoproteins). The inhibition of α-glucosidases by miglustat was shown to disrupt the proper folding of Spike during its maturation from the endoplasmic reticulum to the cell surface, leading to a marked decrease in viral proteins and a reduction in release of infectious virus. It has also previously been shown that iminosugars alter the glycosylation patterns and function of the ACE2 receptor (Zhao et al., 2014).

Although AGIs were first described as antiviral agents in the early 1980's (Pan et al., 1983; Romero et al., 1983) and several AGIs have progressed to antiviral clinical trials, no AGI has been clinically approved for the treatment of viral infections to date. A recent review (Alonzi et al., 2017) detailed the clinical prospects and challenges of iminosugar antivirals (i.e., AGIs), indicating that while a variety of AGIs, such as miglustat, UV-4 (N-7-oxadecyldeoxynojirimycin) and celgosivir, have been taken into clinical development, all have failed to provide a viable clinical candidate. The reasons for this are varied and include the difficulty in maintaining therapeutic concentrations of the AGIs in serum, unwanted side effects at therapeutic concentrations and/or a lack of clinical efficacy. Further, while pulmonary drug delivery (e.g., by oral or nasal inhalation) for the treatment of RTIs is desirable, predicting the biological outcomes of pulmonary drug delivery from first principles has been challenging (Hickey, 2020). Delivery of drug molecules into the respiratory tract and the lower lungs to provide a therapeutic effect can also be challenging.

Accordingly, there is an ongoing need for improved or alternative methods for treating or preventing respiratory tract infections, such as SARS-CoV-2.

### SUMMARY

It is now considered that α-glucosidase inhibitors, and pharmaceutical compositions comprising the same, may be suitable for pulmonary administration and provide an alternative method for treating respiratory tract infections.

In one aspect, the present invention provides a method for treating or preventing a respiratory tract infection in a subject comprising pulmonary administration of a therapeutically effective amount of an α-glucosidase inhibitor to the subject.

In another aspect, the present invention provides use of an α-glucosidase inhibitor in the manufacture of a medicament for treating or preventing a respiratory tract infection in a subject, wherein treating or preventing comprises pulmonary administration of the α-glucosidase inhibitor to the subject.

In another aspect, the present invention provides an α-glucosidase inhibitor for use in treating or preventing a respiratory tract infection in a subject by pulmonary administration.

In yet another aspect, the present invention provides an inhalable composition comprising an α-glucosidase inhibitor and a pharmaceutically acceptable carrier, diluent, adjuvant or excipient.

In accordance with the above aspects of the invention, the α-glucosidase inhibitor may be a compound of general formula (I) or a stereoisomer thereof:

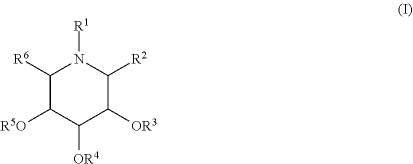

or a pharmaceutically acceptable salt thereof,
wherein:
R¹ is selected from H, hydroxy, C₁₋₁₀alkyl optionally substituted with one or more
R^{X} groups, C₂₋₁₀alkenyl optionally substituted with one or more R^{X} groups, C₂₋₁₀alkynyl optionally substituted with one or more R^{X} groups, or LR⁹; L is a divalent linker group selected from C₁₋₁₀alkyl-O— or C₁₋₁₀alkyl-N R⁷—;
R⁹ is selected from C₁₋₆alkyl optionally substituted with one or more R^{X} groups, R⁸, C₃₋₁₀cycloalkyl optionally substituted with one or more R^{X} groups, C₅₋₁₀ocycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁₋₉heteroaryl optionally substituted with one or more R^{X} groups;
R² is selected from H, hydroxy or OC₁₋₆alkyl;
R², R³, R⁴ and R⁵ are independently selected from H, C₁₋₆alkyl, C₁₋₆alkyl-OH or C(O)C₁₋₆alkyl;
R⁶ is selected from H, hydroxy or C₁₋₇alkyl-OH;
R⁷ and R⁸ are independently selected from H, C₁₋₆alkyl optionally substituted with one or more R^{X} groups, CC₃₋₁₀cycloalkyl optionally substituted with one or more R^{X} groups, or C(O)NH—C₁₋₆alkyl;
or wherein either R¹ and R⁶ or R² and R⁶ together with the atoms to which they are attached form a 5 or 6 membered heterocycloalkyl ring optionally substituted with one or more R^{X} groups; and
each R^{X} is independently selected from hydroxy, halo, nitro, azido, C₁₋₆alkyl, C₂₋₆alkenyl, C₂₋₆alkynyl, C₁₋₆alkoxy, C₃₋₁₀ocycloalkyl, C₅₋₁₀cycloalkenyl, C₂₋₆heterocycloalkyl, C₆₋₁₀aryl, CC₁₋₉heteroaryl or CO(O)C₁₋₆alkyl.

In some embodiments, the α-glucosidase inhibitor may be a compound of general formula (IA):

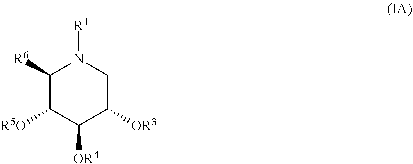

or a pharmaceutically acceptable salt thereof,
wherein R¹, R³, R⁴, R⁵ and R⁶ are as defined above for general formula (I).

In other embodiments, the α-glucosidase inhibitor may be a compound of general formula (IB) or (IC):

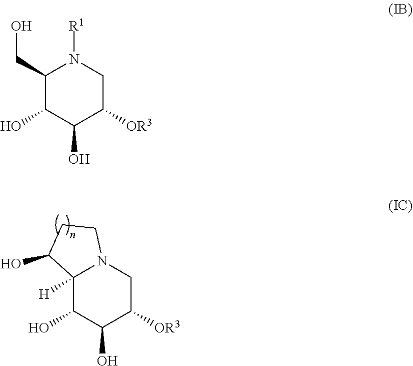

or a pharmaceutically acceptable salt thereof,
wherein R¹ and R³ are as defined above for general formula (I), and n is 1 or 2.

In preferred embodiments, the α-glucosidase inhibitor may be a compound of general formula (ID):

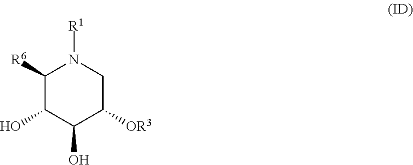

or a pharmaceutically acceptable salt thereof,
wherein:
R¹ is C₁₋₁₀alkyl optionally substituted with one or more R^{X} groups, or LR⁹;
L is a divalent linker group selected from C₁₋₁₀alkyl-O—, or C₁₋₁₀alkyl-NR⁷—;
R⁹ is selected from C₁₋₄alkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl, or C₁₋₉heteroaryl group optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆-loaryl optionally substituted with one or more R^{X} groups, or C₁₋₉heteroaryl optionally substituted with one or more R^{X} groups;
R³ is H or C(O)C₁₋₆alkyl;
R⁶ is CH₂—OH;
R⁷ and R⁸ are independently selected from H, C₁₋₄alkyl, C₃₋₆cycloalkyl, or C(O)NH—C₁₋₄alkyl;
or wherein R¹ and R⁶ together with the atoms to which they are attached form a 5-membered heterocycloalkyl ring substituted with a hydroxyl group; and
each R^{X} is independently selected from hydroxy, halo, nitro, azido, C₃₋₁₀cycloalkyl, C₁₋₄alkoxy and CO(O)C₁₋₄alkyl.

In preferred embodiments of the compound of Formula (ID), R¹ is C₁₋₆alkyl optionally substituted with one or more R^{X} groups or LR⁹.

In preferred embodiments of the compound of Formula (ID), L is a divalent linker group selected from C₁₋₆alkyl-O—, or C₁₋₆alkyl-NR⁷—.

In preferred embodiments of the compound of Formula (ID), each R^{X} is independently selected from hydroxy, halo, nitro and azido.

In other embodiments, the α-glucosidase inhibitor may be:

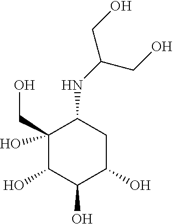 [SMILES: OCC(CO)N[C@@H]1C[C@H](O)[C@@H](O)[C@H](O)[C@@]1(O)CO]

or a pharmaceutically acceptable salt thereof.

In other embodiments, the α-glucosidase inhibitor may be:

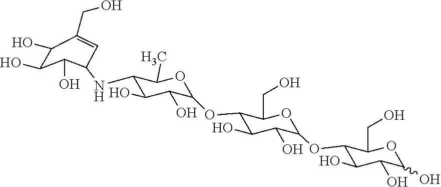 [SMILES: CC1OC(OC2C(CO)OC(OC3C(CO)OC(O)C(O)C3O)C(O)C2O)C(O)C(O)C1NC1C=C(CO)C(O)C(O)C1O]

or a pharmaceutically acceptable salt thereof.

In another aspect, the present invention provides a method for treating or preventing a viral respiratory tract infection in a subject comprising pulmonary administration of an α-glucosidase inhibitor to the subject and wherein the α-glucosidase inhibitor is of a compound of Formula (I), (IA), (TB), (IC) or (ID) as defined above, or a pharmaceutically acceptable salt thereof. In preferred embodiments, the viral infection is a coronavirus infection, preferably SARS-CoV-2.

In another aspect, the present invention provides a method for treating or preventing a coronavirus infection, preferably SARS-CoV-2, in a subject comprising pulmonary administration of an α-glucosidase inhibitor to the subject, wherein the α-glucosidase inhibitor is of a compound of Formula (ID) as defined above, or a pharmaceutically acceptable salt thereof.

In some embodiments, the α-glucosidase inhibitor of Formula (ID) is selected from:

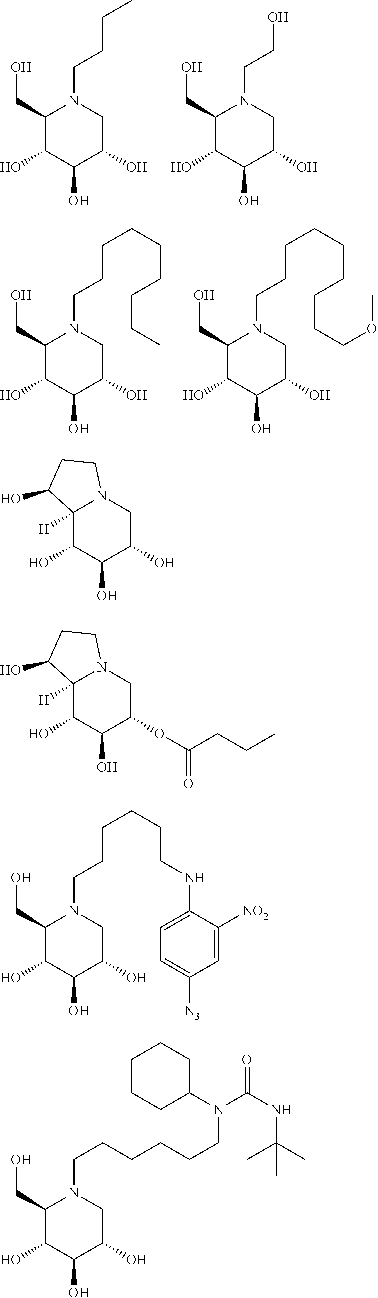

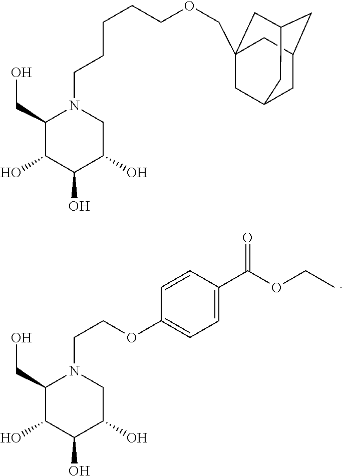 [SMILES: OC[C@@H]1[C@@H](O)[C@H](O)[C@@H](O)CN1CCCCCOCC12CC3CC(CC(C3)C1)C2 ; CCOC(=O)c1ccc(OCCN2C[C@H](O)[C@@H](O)[C@H](O)[C@H]2CO)cc1]

or a pharmaceutically acceptable salt thereof.

In another aspect, the present invention provides a method for treating or preventing a coronavirus infection, preferably SARS-CoV-2, in a subject comprising pulmonary administration of an α-glucosidase inhibitor to the subject, wherein the α-glucosidase inhibitor is:

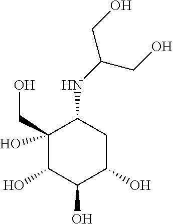 [SMILES: OCC(CO)N[C@@H]1C[C@H](O)[C@@H](O)[C@H](O)[C@@]1(O)CO]

or a pharmaceutically acceptable salt thereof.

### BRIEF DESCRIPTION OF THE DRAWINGS

Embodiments of the invention will now be described with reference to the following Figures, which are intended to be exemplary only, and in which:

FIG. 1 shows a scanning electron microscope (SEM) image of spray dried miglustat (NB-DNJ) aspartate salt (EHT=3.00 kV; WD=10.4 mm).

FIG. 2 shows scanning electron microscope (SEM) images of glyset (miglitol) air jet milled without magnesium stearate (EHT=3.00 kV; WD=12.7 mm).

FIG. 3 shows scanning electron microscope (SEM) images of glyset (miglitol) air jet milled with 5% w/w magnesium stearate (EHT=3.00 kV; WD=12.9 mm).

FIG. 4 shows scanning electron microscope (SEM) images of glyset (miglitol) air jet milled with 3% w/w magnesium stearate followed by mechanofusion (EHT=3.00 kV; WD=10.5 mm).

FIG. 5 shows pressure dispersion test results for miglitol and miglustat co-micronizing with magnesium stearate.

### DEFINITIONS

Unless defined otherwise, all technical and scientific terms used herein have the same meanings as commonly understood by one of ordinary skill in the art to which this invention belongs.

In the context of the present specification, the terms “pulmonary infection” and “respiratory tract infection” are used interchangeably and are intended to have the same meaning.

In the context of the present specification, the terms “composition” and “formulation” are used interchangeably and are intended to have the same meaning.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

As used herein, the term “about” means±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

A reference to a percentage (%) content throughout this specification is to be taken to mean a percentage by weight (wt %). Unless otherwise specified, any reference to a percentage by weight (wt %) of a component of a composition or formulation described herein refers to the wt % of the specified component with respect to the total components of the composition or formulation.

As used herein, the term “alkyl” may refer to either a monovalent (“alkyl”) and divalent (“alkylene”) straight chain or branched chain saturated aliphatic groups. The alkyl group may have from 1 to 10 carbon atoms, denoted C₁₋₁₀alkyl, or it may have from 1 to 6 carbon atoms, denoted C₁₋₆alkyl, or it may have from 1 to 4 carbon atoms, denoted C₁₋₄alkyl. Examples of suitable alkyl groups may include, but are not limited to, methyl, ethyl, 1-propyl, isopropyl, 1-butyl, 2-butyl, isobutyl, tert-butyl, amyl, 1.2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1.3-dimethylbutyl, 1,2,2-trimethylpropyl and 1,1,2-trimethylpropyl, heptyl, octyl, nonyl, decyl and the like.

As used herein, the term “alkenyl” may refer to either a monovalent (“alkenyl”) and divalent (“alkenylene”) straight or branched chain unsaturated aliphatic hydrocarbon groups having at least one double bond anywhere in the chain. Unless indicated otherwise, the stereochemistry about each double bond may be independently cis or trans, or E or Z, as appropriate. The alkenyl group may have from 2 to 10 carbon atoms, denoted C₂₋₁₀alkenyl, or it may have from 1 to 6 carbon atoms, denoted C₂₋₆alkenyl, or it may have from 1 to 4 carbon atoms, denoted C₂₋₄alkenyl. Examples of suitable alkenyl groups may include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl and 2-methylpentenyl.

As used herein, the term “alkynyl” may refer to either monovalent (“alkynyl”) and divalent (“alkynylene”) straight or branched chain unsaturated aliphatic hydrocarbon groups having at least one triple bond. The alkynyl group may have from 2 to 10 carbon atoms, denoted C₂₋₁₀alkynyl, or it may have from 2 to 6 carbon atoms, denoted C₂₋₆alkynyl, or it may have from 2 to 4 carbon atoms, denoted C₂₋₄alkynyl. Examples of suitable alkynyl groups may include, but are not limited to, ethynyl, 1-propynyl, 1-butynyl, 2-butynyl, 1-methyl-2-butynyl, 3-methyl-1-butynyl, 1-pentynyl, 1-hexynyl and methylpentynyl.

As used herein, the term “alkoxy” refers to straight chain or branched alkoxy (O-alkyl) groups, wherein alkyl is as defined above. Examples of suitable alkoxyl groups may include, but are not limited to, methoxy, ethoxy, n-propoxy, isopropoxy, sec-butoxy, and tert-butoxy.

As used herein, the term “aryl” refers to an optionally substituted monocyclic, or fused polycyclic, aromatic carbocycle (i.e., a ring structure having ring atoms that are all carbon). The aryl group may have from 6-10 atoms per ring, denoted C₆₋₁₀aryl. Examples of suitable aryl groups may include, but are not limited to, phenyl, naphthyl, phenanthryl As used herein, the term “aryl” is also intended to encompass optionally substituted partially saturated bicyclic aromatic carbocyclic moiety in which a phenyl and a cycloalkyl or cycloalkenyl group are fused together to form a cyclic structure, such as tetrahydronaphthyl, indenyl or indanyl. The aryl group may be a terminal group or a bridging group.

As used herein, the term “cycloalkyl” refers to a saturated or partially saturated, monocyclic, fused or spiro polycyclic, carbocycle. The cycloalkyl group may have from 3 to 10 carbon atoms per ring, denoted C₃₋₁₀cycloalkyl. Examples of suitable cycoalkyl groups may include, but are not limited to, cyclopropyl, cyclobutyl, cyclopentyl, cyclohexyl, spiro[3.3]heptanyl, decalin and adamantyl. The cycloalkyl group may be a terminal group or a bridging group.

As used herein, the term “cycloalkenyl” refers to a non-aromatic monocyclic or multicyclic ring system containing at least one carbon-carbon double bond. The cycloalkyl group may have from 5 to 10 carbon atoms per ring, denoted C₅₋₁₀cycloalkenyl. Examples of suitable cycloalkenyl groups may include, but are not limited to, include cyclopentenyl, cyclohexenyl and cycloheptenyl. The cycloalkenyl group may be a terminal group or a bridging group.

As used herein, the terms “halogen” or “halo” are interchangeable and refer to fluorine, chlorine, bromine or iodine.

As used herein, the term “heterocycloalkyl” refers to a saturated or partially saturated, monocyclic, bicyclic, fused or spiro polycyclic carbocycles, wherein at least one (e.g., 1, 2, 3, 4 or 5) ring atom is a heteroatom independently selected from O, N, NH, or S. The heterocycloalkyl group may have from 2 to 6 carbon atoms per ring, denoted C₂₋₆heterocycloalkyl. Examples of suitable heterocycloalkyl groups may include, but are not limited to, aziridinyl, azetidinyl, pyrrolidinyl, piperidinyl, piperazinyl, quinuclidinyl, morpholinyl, diazaspiro [3.3]heptane (e.g., 2,6-diazaspiro [3.3]teptane), tetrahydrothiophenyl, tetrahydrofuranyl and tetrahydropyranyl. The heterocycloalkyl group may be a terminal group or a bridging group and may be attached through a heteroatom or any carbon ring atom.

As used herein, the term “heteroaryl” refers an optionally substituted monocyclic, or fused polycyclic, aromatic heterocycle, wherein at least one (e.g., 1, 2, 3, 4, 5, 6, 7 or 8) ring atom is a heteroatom independently selected from O, N, NH, or S. The heteroaryl group may have from 1-9 carbon atoms per ring, denoted C₁₋₉heteroaryl. Examples of suitable heteroaryl groups include, but are not limited to, furyl, imidazolyl, isoxazolyl, isothiazolyl, oxadiazolyl, oxazolyl (e.g., 1,3-oxazolyl, 1,2-oxazolyl), pyridinyl (e.g., 2-, 3-, 4-pyridinyl), pyridazinyl, pyrimidinyl, pyrazinyl, pyrazolyl, pyrrolyl, tetrazolyl, thiadiazolyl, thiazolyl, thienyl, triazolyl (e.g., 1,2,3-triazolyl, 1,2,4-triazolyl), and triazinyl. Representative examples of bicyclic heteroaryl include, but are not limited to, benzimidazolyl, benzofuranyl, benzothienyl, benzoxadiazolyl (e.g., 2,1,3-benzoxadiazolyl), cinnolinyl, dihydroquinolinyl, dihydroisoquinolinyl, furopyridinyl, indazolyl, indolyl (e.g, 2- or 3-indolyl), isoquinolinyl (e.g., 1-, 3-, 4-, or 5-isoquinolinyl), naphthyridinyl (e.g., 1,5-naphthyridinyl, 1,7-naphthyridinyl, 1 ,8-naphthyridinyl, etc), pyrrolopyridinyl (e.g., pyrrolo[2,3-b]pyridinyl), quinolinyl (e.g., 2-, 3-, 4-, 5-, or 8-quinolinyl), quinoxalinyl, tetrahydroquinolinyl, and thienopyridinyl. In one or more embodiments the heteroaryl group is an N-heteroaryl group having one or more nitrogen heteroatoms, e.g., 1, 2, 3 or 4 nitrogen heteroatoms depending on the particular structure. N-heteroaryl groups may also have heteroatoms other than nitrogen, but N-heteroaryl groups are characterized by having at least one nitrogen heteroatom. Exemplary N-heteroaryl groups include imidazolyl, indolyl, (e.g., 2- or 3- indolyl), naphthyridinyl, pyrazinyl, pyridyl (e.g., 2-, 3- or 4-pyridyl), pyrrolyl, pyrimidinyl, quinolinyl (e.g., 2-, 3-, 4-, 5-, or 8-quinolinyl), isoquinolinyl, quinazolinyl, quinoxalinyl and triazinyl. As used herein, the term “heteroaryl” is also intended to encompass optionally substituted partially saturated bicyclic aromatic heterocyclic moiety in which a heterocycle and a cycloalkyl or cycloalkenyl group are fused together to form a cyclic structure. The heteroaryl group may be a terminal group or a bridging group and may be attached through a heteroatom or any carbon ring atom.

As used herein, the term “optionally substituted” when used with reference to a particular group refers means that group may or may not be further substituted or fused (so as to form a polycyclic system), with one or more non-hydrogen substituent groups. Suitable optional substituents will be apparent to those skilled in the art. Exemplary optional substituents may include, but are not limited to, hydroxy, halo, nitro, azido, C₁₋₆alkyl, C₂₋₆alkenyl, C₂₋₆alkynyl, C₁₋₆alkoxy, C₃₋₁₀cycloalkyl, C₅₋₁₀cycloalkenyl, C₂₋₆heterocycloalkyl, C₆₋₁₀aryl, C₁₋₉heteroaryl or CO(O)C₁₋₆alkyl.

The reference to any prior art in this specification is not, and should not be taken as, an acknowledgment or any form of suggestion that that prior art forms part of the common general knowledge.

### DETAILED DESCRIPTION

The present invention relates to α-glucosidase inhibitors (AGIs), compositions comprising the same, and their use in treating or preventing pulmonary diseases, particularly pulmonary infections. In particular, the present invention is directed to use of AGIs, and certain pharmaceutical compositions comprising the same, for treating or preventing bacterial or viral infections of the respiratory tract (i.e., pulmonary infections) by pulmonary administration. Certain AGIs disclosed herein may be particularly suitable for use in the treatment of viral respiratory tract infections, particularly coronavirus-related infections such as SARS-CoV-2, when administered via the pulmonary route.

As used herein, the term “pulmonary administration” when used in relation to an AGI refers to any mode of administration that involves introducing the AGI directly into the respiratory tract (including the upper and/or lower respiratory tract). In particular, pulmonary administration may involve oral inhalation, nasal inhalation, or both. Pulmonary administration may be particularly advantageous because it can enable targeting of the drug delivery directly into the lungs for both local and systemic treatment. Preferably, the AGI is administered via oral inhalation. Nasal administration for local AGI delivery may, for example, be limited by the size of a subjects nasal passages. Further, the concentration loss of small particles administered by oral inhalation can be significantly less at the active site than the corresponding particles administered via nasal inhalation.

As previously described in WO 2013/016754, significant technological barrier to preparing inhalable formulations for the treatment of RTIs has been the practicality of engineering a drug containing aerosol suitable for highly efficient delivery (e.g., >50% dose delivery to site of treatment), reproducible delivery (e.g., having a coefficient of variation (CV%) of dose delivery <10%) and a high payload (e.g., >1 mg powder delivery to site of treatment) in a practical and cost effective manner. While dry powders present an attractive means of drug delivery, generating micronized particles suitable for highly efficient aerosolisation has presented a significant technical challenge. Upon inhalation, larger aerosolised drug particles tend to be deposited by impaction and gravitational sedimentation at the back of the throat and upper respiratory tract where they are prone to mucociliary clearance into the gastro intestinal tract and subsequent metabolism. Also, larger drug particles cannot progress deep into the lower lung due to the narrowing of the bronchioles. It has previously been postulated that for effective local aerosol transport and delivery to the respiratory system including trachea, bronchi and alveoli, particles of less than 5 μm aerodynamic diameter may be preferred, while for deep lung, bronchioles and alveoli, particles of less than 3 μm may be preferred.

For the purposes of targeted inhaled delivery, it may be preferable to provide an inhalable aerosol with an aerodynamic diameter size distribution that targets both upper and lower sites in the respiratory tract. In this regard, inhaled aerosol delivery as an aerosolised dry powder may provide an attractive delivery format. However, generating micronized particles suitable for highly efficient aerosolisation can be challenging due to the inherent cohesion of drug particles that are smaller than 5 μm aerodynamic diameter as required for targeted aerosol delivery. Added to this challenge is the real world variation presented by the unique interfacial properties of individual drugs substances, which may include variations across different batches of the same substance.

In addition to the problems mentioned above, it is particularly advantageous for any practical pulmonary delivery system for dry powders to avoid or minimise agglomeration of the particles at the time of administration, have low variation in delivered dose due to poor flow properties or inconsistent agglomeration and/or avoid or minimise incomplete removal of the powder from the delivery device caused by adhesion of powder to the walls of the device.

There are also significant technological challenges associated with the development of other inhalable formulations, such as liquid or suspension formulations for inhalation, which include balancing such variables as the physico-chemical properties of the drug substance and any propellants, surfactants and other components of the composition and their interaction with each other and/or the device used for administration.

It is now considered that pulmonary administration of AGIs, or compositions comprising the same may be suitable for the treatment of respiratory tract infections. In particular, delivery of AGI formulations by inhalation administration may be suitable for maintaining therapeutic concentrations and avoiding dose limiting side effects observed with oral administration of AGIs.

### Alpha-Glucosidase Inhibitors

Alpha-glucosidases, such as maltase, dextranase, sucrase and glucoamylase, are enzymes responsible for breaking down complex carbohydrates into glucose, which can be absorbed into the bloodstream. Alpha-glucosidase inhibitors (AGIs) are carbohydrate analogues that bind reversibly to α-glucosidases. As used herein, the term “glucosidase inhibitor” includes any agent that decreases, inhibits or impairs at least one function or biological activity of a glucosidase. For example, as used herein, the terms “alpha-glucosidase inhibitor”, “α-glucosidase inhibitor” and “AGI” are interchangeable and refer to an agent that decreases, inhibits or impairs at least one function or biological activity of α-glucosidase, including α-glucosidase I and II.

In particular, in seeking an expeditious treatment for RTIs, particularly SARS-CoV-2, AGIs that have previously been investigated for therapeutic use may be particularly suitable. Preferably, the AGIs have been approved for use in humans. Exemplary AGIs previously investigated for therapeutic use include acarbose (approved for treatment of diabetes mellitus type 2), miglustat (NB-DNJ; approved for treatment of type I Gaucher disease), glyset (miglitol; approved for treatment of diabetes mellitus type 2), emiglitate (BAY1248; inhibitor of glucose-induced insulin release), N-nonyldeoxynojirimycin (NNDNJ; inhibitor and pharmacological chaperone of lysosomal β-glucosidase), N-7-oxadecyldeoxynojirimycin (UV-4; SP 116; in vitro and in vivo dengue antiviral activity), voglibose (approved for lowering post-prandial blood glucose levels in people with diabetes mellitus), castanospermine (in vitro antiviral activity), celgosivir (6-O-butanoylcastanospermine; hepatitis C virus (HCV) infection), among others. Other known AGIs that may be suitable for use in humans include NAP-DNJ (N-(6′-[4″-azido-2″-nitrophenylamino]hexyl)-1-deoxynojirimycin), IHVR- 19029 (3-tert-butyl-1-cyclohexyl-1-[6-[(2R,3R,4R,5S)-3,4,5-trihydroxy-2-(hydroxymethyl)piperidin-1-yl]hexyl]urea) and AMP-DNM (N-adamantanemethyloxypentyl-1-deoxynojirimycin), among others. However, as previously mentioned, AGIs have previously failed to provide a viable clinical candidate for the treatment of RTIs and administration of AGIs by inhalation has not previously been explored.

By way of example, miglustat (marketed as Zavesca® and also known as N-butyl-1-deoxynojirimycin, NB-DNJ) is an AGI that has been approved in many jurisdictions for the treatment of Gaucher and Niemann Pick disease. Miglustat was originally identified and developed as an anti-HIV agent and exhibited promising antiviral effects in Phase II trials in combination with AZT (zidovudine). However, further dose escalation trials using miglustat as an HIV monotherapy were halted due to fears of toxicity (which were ultimately unfounded) and development of the investigational drug was halted for HIV. Miglustat has also been investigated as an antiviral agent for a number of other viruses and has progressed to Phase II/III clinical trials but is not yet approved as an antiviral. Miglustat is delivered via the oral route in 100 mg capsules. The dose for Gaucher disease is 100-300 mg t.i.d. for long-term chronic treatment, although higher doses were used in the HIV trials and in safety studies. Miglustat has been reported to be active against SARS-CoV-1 in vitro with an EC₅₀ of ˜100 μg/ml, which is analogous to the effective concentrations reported for HIV-1 in vitro. It has also been reported to be very well tolerated by cells in culture and has little cytotoxicity.

It is now considered that pulmonary delivery of AGIs may provide an alternative method for treating respiratory tract infections (RTIs), particularly when formulated as dry powders for inhalation. Suitable α-glucosidase inhibitors will be known to those skilled in the art and may include, but are not limited to, acarbose, miglustat (NB-DNJ), glyset (miglitol), emiglitate (BAY1248), N-nonyldeoxynojirimycin (NNDNJ), N-7-oxadecyldeoxynojirimycin (UV-4; SP 116), voglibose, castanospermine, celgosivir (6-O-butano ylcastanospermine), NAP-DNJ (N-(6′-[4″-azido-2″-nitrophenylamino]hexyl)-1-deoxynojirimycin), IHVR-19029 ((3-tert-butyl-1-cyclohexyl-1-[6-[(2R,3R,4R,5S)-3,4,5-trihydroxy-2-(hydroxymethyl)piperidin-1-yl]hexyl]urea) and AMP-DNM (N-adamantanemethyloxypentyl-1-deoxynojirimycin).

It will be recognized that the AGIs disclosed herein may possess asymmetric centers and are therefore capable of existing in more than one stereoisomeric form. Thus, the AGIs disclosed herein may exist as single stereoisomers, racemates, and/or mixtures of enantiomers and/or diastereomers. Thus, unless otherwise specified, any reference to an AGI herein includes stereoisomers thereof. As used herein, the term “stereoisomer” refers to any two or more isomers that have the same molecular constitution and differ only in the three dimensional arrangement of their atomic groupings in space. Stereoisomers may be diastereoisomers or enantiomers. In some embodiments, the AGIs disclosed herein may be in substantially pure isomeric form at one or more asymmetric centers (e.g., greater than about 90% ee, 95% ee, 97% ee or 99% ee), or a mixture (including racemic mixtures) thereof.

A number of known AGIs, such as miglustat (NB-DNJ), glyset (miglitol), emiglitate (BAY1248), N-Nonyldeoxynojirimycin (NNDNJ), N-7-oxadecyldeoxynojirimycin (UV-4; SP 116), castanospermine, celgosivir (6-O-butanoylcastanospermine), NAP-DNJ, IHVR-19029 and AMP-DNM, each share a common iminosugar structural motif. Accordingly, pulmonary administration of iminosugar derivatives may be useful for the treatment or prevention of RTIs.

Accordingly, AGIs suitable for use in the present invention may include compounds of general formula (I) or a stereoisomer thereof:

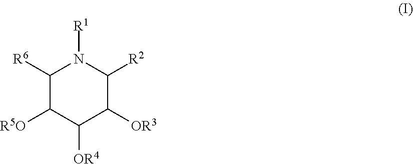

or a pharmaceutically acceptable salt thereof,
wherein:
R¹ is selected from H, hydroxy, C₁₋₁₀alkyl optionally substituted with one or more R^{X} groups, C₂₋₁₀alkenyl optionally substituted with one or more R^{X} groups, C₂₋₁₀alkynyl optionally substituted with one or more R^{X} groups, or LR⁹;
L is a divalent linker group selected from C₁₋₁₀alkyl-O- or C₁₋₁₀alkyl-N R⁷—;
R⁹ is selected from C₁₋₆alkyl optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁₋₉heteroaryl optionally substituted with one or more R^{X} groups;
R² is selected from H, hydroxy or OC₁₋₆alkyl;
R², R³, R⁴ and R⁵ are independently selected from H, C₁₋₆alkyl, C₁₋₆alkyl-OH or C(O)C₁₋₆alkyl;
R⁶ is selected from H, hydroxy or C₁₋₆alkyl-OH;
R⁷ and R⁸ are independently selected from H, C₁₋₆alkyl optionally substituted with one or more R^{X} groups, C₃₋₁₀cycloalkyl optionally substituted with one or more R^{X} groups, or C(O)NH—C₁₋₆alkyl;
or wherein either R¹ and R⁶ or R² and R⁶ together with the atoms to which they are attached form a 5 or 6 membered heterocycloalkyl ring optionally substituted with one or more R^{X} groups; and
each R^{X} is independently selected from hydroxy, halo, nitro, azido, C₁₋₆alkyl, C₂₋₆alkenyl, C₂₋₆alkynyl, C₁₋₆alkoxy, C₃₋₁₀ocycloalkyl, C₅₋₁₀Cycloalkenyl, C₂₋₆heterocycloalkyl, C₆₋₁₀aryl, C₁₋₉heteroaryl or CO(O)C₁₋₆alkyl.

In preferred embodiments of the AGI or formula (I), R² is H.

In preferred embodiments, the α-glucosidase inhibitors are of general formula (IA):

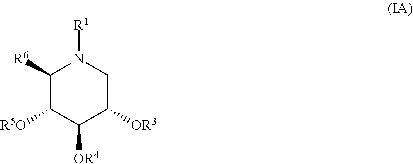

or a pharmaceutically acceptable salt thereof,
wherein:
R¹ is selected from H, hydroxy, C₁₋₁₀alkyl optionally substituted with one or more R^{X} groups, C₂₋₁₀alkenyl optionally substituted with one or more R^{X} groups, C₂₋₁₀alkynyl optionally substituted with one or more R^{X} groups, or LR⁹;
L is a divalent linker group selected from C₁₋₁₀alkyl-O— or C₁₋₁₀alkyl-NR⁷—;
R⁹ is selected from C₁₋₆alkyl optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁₋₉heteroaryl optionally substituted with one or more R^{X} groups; R³, R⁴ and R⁵ are independently selected from H, C₁₋₆alkyl, C₁₋₆alkyl-OH or C(O)C₁₋₆alkyl;
R⁶ is selected from H, hydroxyl or C₁₋₆alkyl-OH,
R⁷ and R⁸ are independently selected from H, C₁₋₆alkyl optionally substituted with one or more R^{X} groups, C₃₋₁₀cycloalkyl optionally substituted with one or more R^{X} groups, or C(O)NH—C₁₋₆alkyl;
or wherein either R¹ and R⁶ or R² and R⁶ together with the atoms to which they are attached form a 5 or 6 membered heterocycloalkyl ring optionally substituted with one or more R^{X} groups; and
each R^{X} is independently selected from hydroxy, halo, nitro, azido, C₁₋₆alkyl, C₂₋₆alkenyl, C₂₋₆alkynyl, C₁₋₆alkoxy, C₃₋₁₀cycloalkyl, C₅₋₁₀cycloalkenyl, C₂₋₆heterocycloalkyl, C₆₋₁₀aryl, C₁₋₉heteroaryl or CO(O)C₁₋₆alkyl.

In preferred embodiments of the AGI of general formulae (I) and/or (IA), R³, R⁴ and R⁵ each H.

In preferred embodiments of the AGI of general formulae (I) and/or (IA), R⁶ is CH₂—OH.

In preferred embodiments of the AGI of general formulae (I) and/or (IA), R³, R⁴ and R⁵ each H and R⁶ is CH2-OH.

In preferred embodiments of the AGI of general formulae (I) and/or (IA), R¹ and R⁶, together with the atoms to which they are attached, form a 5-membered heterocycloalkyl ring optionally substituted with one or more R^{X} groups. Preferably, R^{X} is hydroxy.

In particularly preferred embodiments, the α-glucosidase inhibitors are of general formula (TB) or (IC):

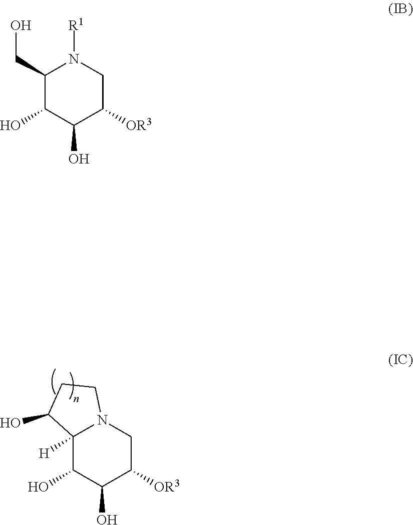

or a pharmaceutically acceptable salt thereof,
wherein:
R¹ is selected from H, hydroxy, C₁₋₁₀alkyl optionally substituted with one or more R^{X} groups, C₂₋₁₀alkenyl optionally substituted with one or more R^{X} groups, C₂₋₁₀alkynyl optionally substituted with one or more R^{X} groups, or LR⁹;
L is a divalent linker group selected from C₁₋₁₀alkyl-O— or C₁₋₁₀alkyl-NR⁷—;
R⁹ is selected from C₁₋₆alkyl optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁₋₉heteroaryl optionally substituted with one or more R^{X} groups;
R³ is selected from H, C₁₋₆alkyl or C(O)C₁₋₆alkyl;
R⁷ and R⁸ are independently selected from H, C₁₋₆alkyl optionally substituted with one or more R^{X} groups, C₃₋₁₀cycloalkyl optionally substituted with one or more R^{X} groups, or C(O)NH—C₁₋₆alkyl;
each R^{X} is independently selected from hydroxy, halo, nitro, azido, C₁₋₆alkyl, C₂₋₆alkenyl, C₂₋₆alkynyl, C₁₋₆alkoxy, C₃₋₁₀ycloalkyl, C₅₋₁₀cycloalkenyl, C₂₋₆heterocycloalkyl, C₆₋₁oaryl, C₁₋₉heteroaryl or CO(O)C₁₋₆alkyl; and n is 1 or 2.

In preferred embodiments of the AGI of general formulae (I), (IA) and/or (TB), R¹ is hydroxy or C₁₋₁₀alkyl optionally substituted with one or more R^{X} groups. Preferably, R^{X} is hydroxy. In other embodiments, R¹ is LR⁹, wherein L is C₁₋₆alkyl-O— or C₁₋₆alkyl-NH— and R⁹ is phenyl optionally substituted with one or two R^{X} groups.

In preferred embodiments of the AGI of general formula (IC), n is 1.

In preferred embodiments of the AGI of general formulae (I), (IA), (IB) and/or (IC), R¹ is LR⁹. Preferably, L is C₁₋₆alkyl-O— or C₁₋₆alkyl-NR⁷—. Preferably, R⁹ is C₁₋₄alkyl (e.g., methyl, ethyl, n-propyl, isopropyl, n-butyl, sec-butyl or t-butyl) optionally substituted with one or more R^{X} groups, phenyl optionally substituted with one or more R^{X} groups or R⁸.

In preferred embodiments of the AGI of general formulae (I), (IA), (IB) and/or (IC), R³ is H or C(O)C₁₋₆alkyl. Preferably, the C(O)C₁₋₆alkyl is C(O)C₁₋₄alkyl, such as C(O)-methyl,

C(O)-ethyl, C(O)-n-propyl, C(O)-iso-propyl, C(O)-n-butyl, C(O)-sec-butyl or C(O)-tert-butyl.

In preferred embodiments of the AGI of general formulae (I), (IA), (IB) and/or (IC), R^{X} may be C₁₋₄alkyl (e.g., methyl, ethyl, n-propyl, isopropyl, n-butyl, sec-butyl or t-butyl). In other preferred embodiments, R^{X} may be independently selected from hydroxy, nitro or azido. In other embodiments, R^{X} may be adamantyl. In yet other preferred embodiments, R^{X} may be a methyl, ethyl, n-propyl, isopropyl, n-butyl, sec-butyl or t-butyl ester.

In a particularly preferred embodiment, the α-glucosidase inhibitors are of Formula (ID):

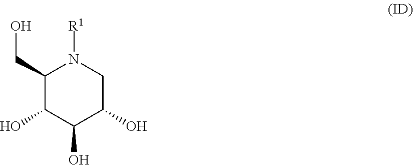

or a pharmaceutically acceptable salt thereof,
wherein:
R¹ is C₁₋₁₀alkyl optionally substituted with one or more R^{X} groups, or LR⁹; L is a divalent linker group selected from C₁₋₁₀alkyl-O—, or C₁₋₁₀alkyl-NR⁷—;
R⁹ is selected from C₁₋₁₄alkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl, or C₁₋₉heteroaryl group optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆-loaryl optionally substituted with one or more R^{X} groups, or C₁₋₉heteroaryl optionally substituted with one or more R^{X} groups;
R³ is H or C(O)C₁₋₆alkyl;
R⁶ is CH₂—OH;
R⁷ and R⁸ are independently selected from H, C₁₋₄alkyl, C₃₋₆cycloalkyl, or C(O)NH—C₁₋₄alkyl;
or wherein R¹ and R⁶ together with the atoms to which they are attached form a 5-membered heterocycloalkyl ring substituted with a hydroxyl group; and
each R^{X} is independently selected from hydroxy, halo, nitro, azido, C₃₋₁₀cycloalkyl, C₁₋₄alkoxy and CO(O)C₁₋₄alkyl.

In preferred embodiments of the compound of Formula (ID), R¹ is C₁₋₆alkyl optionally substituted with one or more R^{X} groups or LR⁹.

In preferred embodiments of the compound of Formula (ID), L is a divalent linker group selected from C₁₋₆alkyl-O—, or C₁₋₆alkyl-NR⁷—.

In preferred embodiments of the compound of Formula (ID), each R^{X} is independently selected from hydroxy, halo, nitro, azido and C₃₋₁₀cycloalkyl.

In a preferred embodiment of the AGI of general formula (ID), R¹ is C₁₋₁₀alkyl optionally substituted with a hydroxy group. Preferably, R¹ is C₁₋₆alkyl optionally substituted with a hydroxy group.

In other preferred embodiments of the AGI of general formula (ID), L is selected from C₁₋₆alkyl-O—, preferably C₄₋₆alkyl-O—, or C₁₋₁₀alkyl-NR⁷—, preferably C₄₋₆alkyl-N R⁷—.

In other preferred embodiments of the AGI of general formula (ID), R¹ is LR⁹ and R⁹ is C₁₋₄alkyl optionally substituted with C₃₋₁₀cycloalkyl. Preferably, L is C₁₋₁₀alkyl-O—, more preferably C₁₋₆alkyl-O—, and R⁹ is C₁₋₂alkyl optionally substituted with C₆₋₁₀cycloalkyl. In a particularly preferred embodiment, R⁹ may be methyl substituted with an adamantyl group.

In other preferred embodiments of the AGI of general formula (ID), R¹ is LR⁹ and R⁹ is R⁸. Preferably, L is C₁₋₁₀alkyl-NR⁷—, more preferably C₁₋₆alkyl-NR⁷—. Preferably, at least one of R⁷ and R⁸ is H or C₃₋₆cycloalkyl (e.g., cyclohexyl). In a particularly preferred embodiment, R⁷ is H and R⁸ is phenyl optionally substituted with one or more R^{X} groups. In another particularly preferred embodiment, R⁷ is C₃₋₆cycloalkyl and R⁸ is C(O)NH—C₁₋₄alkyl.

In a preferred embodiment, the α-glucosidase inhibitor is selected from:

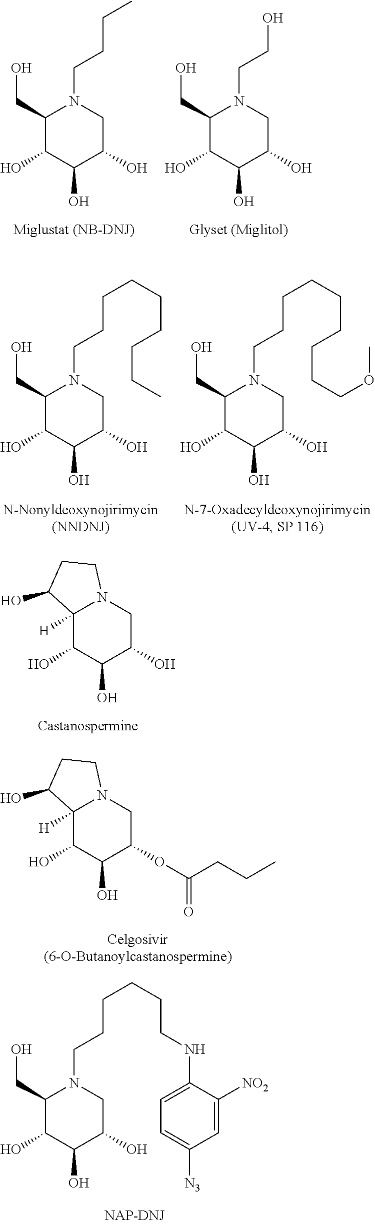

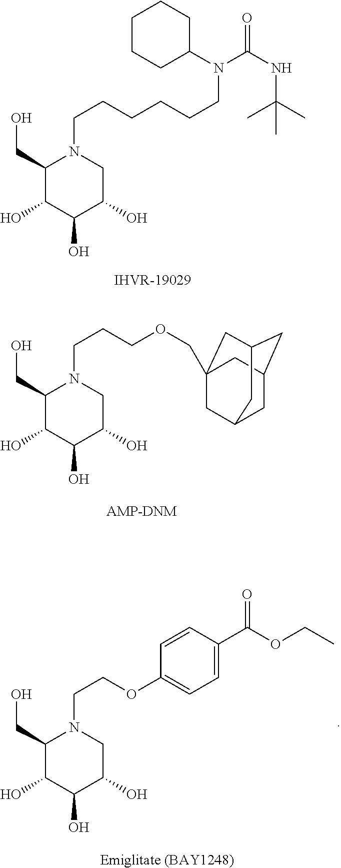

Preferably, the α-glucosidase inhibitor is selected from miglustat (NB-DNJ), glyset (miglitol), NNDNJ, UV-4, castanospermine, celgosivir, NAP-DNJ, IHVR-19029, AMP-DNM and emiglitate, more preferably miglustat (NB-DNJ), glyset (miglitol), NAP-DNJ, IHVR-19029 and AMP-DNM, even more preferably NAP-DNJ and AMP-DNM.

In embodiments of Formulae (I), (IA), (IB), (IC) or (ID) in which a particular group is substituted with one or more R^{X} groups, the number of R^{X} groups is preferably 1 or 2.

Other carbohydrate analogues may also be suitable for use in the present invention. Thus, in another preferred embodiment, an AGI that may be suitable for use in the present invention is:

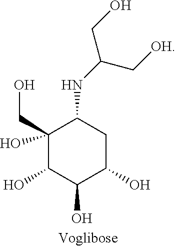 [SMILES: OCC(CO)N[C@@H]1C[C@H](O)[C@@H](O)[C@H](O)[C@@]1(O)CO]

In yet another preferred embodiment, an AGI that may be suitable for use in the present invention is:

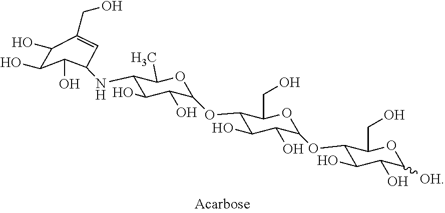 [SMILES: CC1OC(OC2C(CO)OC(OC3C(CO)OC(O)C(O)C3O)C(O)C2O)C(O)C(O)C1NC1C=C(CO)C(O)C(O)C1O]

Other suitable carbohydrate analogues that inhibit α-glucosidase can be readily identified using standard techniques known to those skilled in the art.

AGIs suitable for use in the present invention may be commercially available or prepared according to any suitable method known to those skilled in the art. Stereoisomers of AGIs disclosed herein may be naturally occurring or may be prepared by asymmetric synthesis, for example using chiral intermediates, or by chiral resolution.

### Pharmaceutical Formulations

It is to be understood that the AGIs (including stereoisomers thereof) disclosed herein may be provided as a pharmaceutically salts, hydrates or solvates. The term “pharmaceutically acceptable salts” includes pharmaceutically acceptable solvates and hydrates, and pharmaceutically acceptable addition salts of the AGIs disclosed herein. The term “solvate” includes a molecular complex comprising an AGI or stereoisomer thereof as disclosed herein and one or more pharmaceutically acceptable solvent molecules, for example, ethanol. The term “hydrate” is employed when the solvent is water. It is also contemplated that the AGIs (including stereoisomers thereof) disclosed herein may be suitable for use in veterinary applications. Thus term “pharmaceutically acceptable salts” is also intended to include veterinarilly acceptable solvates and hydrates, and veterinarilly acceptable addition salts of the AGIs disclosed herein.

In some embodiments, AGI salts may include acid addition salts and salts of quaternary amines. For use in medicine, the AGI salts will be pharmaceutically acceptable salts, but it will be appreciated that non-pharmaceutically acceptable salts also fall within the scope of the present invention, since these may be useful as intermediates in the preparation of pharmaceutically acceptable salts. A pharmaceutically acceptable salt involves the inclusion of another molecule such as a chloride ion, an acetate ion, a sulfate ion or other counter ion. The counter ion may be any organic or inorganic moiety that stabilizes the charge on the parent compound. Furthermore, a pharmaceutically acceptable salt may have more than one charged atom in its structure. When multiple charged atoms are present in the parent drug, its pharmaceutically acceptable salts will have multiple counter ions and these can be several instances of the same counter ion or different counter ions. Hence, a pharmaceutically acceptable salt can have one or more charged atoms in the parent compound and/or one or more counter ions.

Acid addition salts may be formed from AGIs and a pharmaceutically acceptable inorganic or organic acid including, but not limited, to hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acids. The counter ions of quaternary amines include, but are not limited to, chloride, bromide, iodide, sulfate, phosphate, methansulfonate, citrate, acetate, malonate, fumarate, sulfamate, and tartrate. Also, basic nitrogen-containing groups may be quaternised with such agents as lower alkyl halides, e.g., methyl, ethyl, propyl, and butyl chlorides, bromides and iodides; dialkyl sulfates like dimethyl and diethyl sulfate; and others. The preparation of the pharmaceutically acceptable salts described above and other typical pharmaceutically acceptable salts is more fully described by Berge et al., “Pharmaceutical Salts,” J. Pharm. Sci., 1977:66:1-19.

In some embodiments, AGI salts may be prepared from the free form of the compound in a separate synthetic step prior to incorporation into the compositions disclosed herein. In still other embodiments, AGI salts may be prepared in situ during preparation of the composition for administration. For example, the composition for administration may further comprise an appropriate acid which, upon contact with the free form of the AGI, forms a desired pharmaceutical salt in situ for administration.

Furthermore, it will recognised by a person skilled in the art that the AGIs disclosed herein may be in crystalline form, either as the free compound or as a solvate (e.g., a hydrate) and it is intended that both forms are within the scope of the present invention. Methods of solvation are generally known within the art.

The present invention also contemplates pharmaceutically acceptable derivatives or prodrugs of the AGIs (including stereoisomers thereof) disclosed herein. For example, the AGI could be provided in the form of a prodrug, which may, upon administration to a subject, be capable of providing (directly or indirectly) the AGIs, or an active metabolite or residue thereof. The term “prodrug” is used in its broadest sense and encompasses those derivatives that are converted in vivo to the active agent (i.e., the AGI). Such derivatives would readily occur to those skilled in the art. By way of non-limiting example, it will be appreciated by those skilled in the art that celgosivir (6-O-butanoylcastanospermine) is a prodrug of castanospermine.

As previously described, the present invention encompasses the use of AGIs as the free base form or as a pharmaceutically salt or solvate thereof in the treatment of pulmonary diseases. Where specific dosages or concentrations of an AGI are referred to herein, it is to be understood that the specific dosage or concentration refers to the concentration of or equivalent to the free base of the AGI. Accordingly, where a pharmaceutically acceptable salt of an AGI is used, a person skilled in the art would readily understand that the concentrations or dosages in respect of the salt, refers to the equivalent concentration or dosage of the free base form of the AGI.

The AGIs disclosed herein, or pharmaceutically acceptable salts thereof, may be administered together with one or more pharmaceutically acceptable carriers, diluents, adjuvants and/or excipients. Where a carrier, diluent, adjuvants and/or excipient is used, they must be “pharmaceutically acceptable” in the sense of being compatible with the other ingredients of the composition and not injurious to the subject. Such pharmaceutically acceptable carriers, diluents, adjuvants or excipients will be apparent to those skilled in the art and may depend on the intended mode of administration. For example, the carriers, diluents, adjuvants or excipients may vary depending on whether the formulation is a dry powder or liquid formulation, whether the formulation is intended for oral or nasal inhalation, and/or, in the case of liquid formulations, whether they are intended for administration as a spray or aerosol.

The AGI formulations used in the present invention are preferably sustained-release formulations for pulmonary administration.

General considerations in the formulation and/or manufacture of pharmaceutical compositions for pulmonary administration, i.e., by oral or nasal inhalation, can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

The AGI's disclosed herein may be administered by any suitable method of pulmonary administration. Pulmonary administration is typically achieved using a pressurized metered dose inhaler, dry powder inhaler or nebulizer. In preferred embodiments, the AGIs disclosed herein are administered by a dry power inhaler or nasal spray. Advantageously, dry powder formulations as disclosed herein may avoid or minimise agglomeration of the particles at the time of administration, have low variation in delivered dose due to poor flow properties or inconsistent agglomeration and/or avoid or minimise incomplete removal of the powder from the delivery device caused by adhesion of powder to the walls of the device.

In other preferred embodiments, the AGIs disclosed herein are administered by a nebulizer, including pressurized metered dose inhalers, which convert liquids into aerosols of a suitable size for inhalation in the respiratory tract. In particular, the pressurized metered dose inhalers may advantageously provide an accurate and consistent dose of the AGI, leading to improved dosing reliability. The preparation of suitable formulations, including the use of suitable propellants, will be within the purview of a person skilled in the art.

In one embodiment, the AGIs disclosed herein may be delivered as an aerosol into the lungs of a subject as a solution in water. The aerosol may be delivered using a pharmaceutically known nebulizer. Suitable nebulizers known in the art include compressed air nebulisers, ultrasonic nebulisers (including vibrating mesh nebulisers) and acoustic wave nebulisers. The nebulisers may include controls to deliver the aerosol only on the inhaled manoeuvre of the patient, or only an optimised part of the inhaled manoeuvre.

Pulmonary administration typically requires the generation of micronized particles. The micronized particles may be administered using a dry powder inhaler or the dry powder may be formulated as a liquid (e.g., a solution or emulsion) for administration by nebulizer or nasal spray. Micronized particles are typically prepared by either milling or by spray drying. Suitable processes and considerations for micronizing particles can be readily determined by the skilled person. In particular, suitable methods for preparing formulations of biologically active agents for delivery by inhalation are disclosed in WO 2013/016754, the entire contents of which are incorporated herein by cross-reference.

In particular, micronized particles may be produced by any suitable form of size reduction. For example, attrition, milling and co-milling may be used to obtain suitably sized particles. Typically, air jet milling may be used, where larger particles of drug are entrained into high speed air jets to induce collisions, which lead to particle fracture. Many forms of air jet mill are known, such as those made by Hosokawa Micron, and may comprise spiral jet mills or fluid bed jet mills. Alternatively other forms of milling such as ball milling or bead milling.

Micronized particles may be formed by any of the known milling processes in the presence of additive materials, in a co-milling process.

It has been found that such industrial micronization can induce amorphous regions on the surfaces of the micronized AGIs disclosed herein. Micronization in such cases occurs via violent impacts on the bulk material in order to cause the required size reduction. It has been observed that even the small amounts of amorphous material present as a result of the mechanical micronization can create physically unstable particles, which rapidly form solid bridges within the powder to prevent aerosolisation. In this situation, it may be advantageous to modify the micronization process by the addition of a second material in the milling step.

Thus, the micronization process may preferably be conducted in the presence of an adhesion modifier, in a co-milling process. Such adhesion modifiers, which may include, but are not limited to, magnesium stearate or other metal stearates, L-leucine or derivatives thereof such as tri-leucine, or other suitable amino acids (e.g., lysine and cysteine), peptides or lipid materials. Preferably, the adhesion modifier is L-leucine or magnesium stearate. Adhesion modifiers may be present in the co-milled composition in an amount between about 0.01 and 10%, preferably about 0.02 and 5%, about 0.02 and 3%, or about 0.1 and 2% by weight relative to the AGI.

In some embodiments, the micronized particles comprise or consist of an AGI. In other embodiments, the micronized particles comprise or consist of an AGI and an adhesion modifier. The micronized particle formulations comprising an AGI as disclosed herein may further include one or more pharmaceutically acceptable carriers, diluents, adjuvants and/or excipients. Other excipients may include, but are not be limited to, bulking agents, buffer agents and stabilisers such as taste or smell modifying agents, adhesion modifiers, flow agents, or dissolution modifiers.

Micronized particles may be obtained and engineered into any known particle engineering system, such as but not limited by the following: Pulmosphere™ or Pulmosol™ technology developed by Nektar, AIR™ porous particle technology developed by Alkermes, Technosphere™ technology developed by Mannkind, Powderhale™ technology developed by Vectura, particles created by Prosonix sonocrystalisation methods, particles created by wet or dry nano-milling technologies for example developed by Elan, Hovione or Savara.

Alternatively, spray drying may be used to produce drug containing particles of the desired micron size range. The term “spray drying” is intended to encompass any process in which a solution of one or more solutes or suspension is formed in a liquid, whereby the liquid is physically atomised into individual droplets, which are then dried to form a dry particulate powder. It may encompass any form of a droplet to particle formation process, and may encompass related processes such as spray-freeze drying, spray chilling and spray drying. The droplets may be formed by any known atomisation process, including but not limited to pressure atomisation, pneumatic atomisation, two or multiple fluid atomisation, rotary disc atomisation, electrohydrodynamic atomisation, ultrasonic atomisation, and any variant of such atomisation processes. The atomisation may occur from one spray source or multiple sources. The liquid vehicle spray may or may not be aqueous and may optionally comprise co-solvents plus additional components dissolved or suspended. Preferably, the liquid includes water. The liquid may employ water alone as a solvent or it may also include one or more organic co-solvents, for example, methanol, ethanol, n-propanol, iso-propanol, acetone, or the like. Any organic solvent used in the liquid should be selected so that it produces a vapour that is significantly below any explosive or combustion limit. The liquid may include a material that is a vapour or solid at ambient conditions but exists as a liquid under the selected process conditions. The droplets formed may be dried by applying heat in the form of a heated drying gas, or heat may be applied in other ways, for example radiatively from the walls of the drying chamber or as microwaves. Once collected from this drying process, the particles may be further dried or conditioned to a controlled moisture level via a process such as vacuum drying or freeze drying. Alternatively drying may be achieved by freezing followed by drying or by application of vacuum. it will be recognised that any other means of obtaining such particles are also contemplated herein, for example super critical fluid synthesis, synthesis from emulsions and any other form of controlled precipitation that forms substantially spherical particles.

Where the particles are spray dried, they may advantageously be co-spray dried with an adhesion modifier such as, magnesium stearate or other metal stearates, L-leucine or derivatives thereof such as tri-leucine, or other peptides or lipid materials. Preferably, the adhesion modifier is L-leucine. The potential advantageous properties provided by adding L-leucine, either by co-milling or by condensation/precipitation, was first demonstrated by Staniforth and Ganderton el al. (See for example, WO 96/23485 and WO 00/33811). This work indicated that peculiar physical properties of this amino acid provided its performance enhancing behaviour. Several groups have since studied the benefit L-leucine provides to powder aerosolization, especially when co-sprayed with actives and excipients, however, the true nature of the structure-performance relationship in such systems remains unclear. More broadly, it also appears that certain peptides/proteins in spray dried particle structures, for example albumins, isoleucine or tri-leucine, may also confer improved aerosolization performance in use. Alternatively, lipid and fatty acid materials may also provide some benefit in this respect, such as phospholipids (for example DPPC), lecithins.

Other suitable materials known to those skilled in the art may also be included in the solution to be co-spray dried.

In some embodiments, it may be necessary to spray dry the AGIs disclosed herein with an adhesion modifier (e.g., L-leucine) in the spray dried solution to prevent the formation of physically unstable highly amorphous particles. The amorphous form of certain AGIs disclosed herein may be highly hygroscopic, so that on precipitation into a solid form, they immediately uptake any moisture present, including the inherent moisture in the atmosphere of the spray dryer, reforming as liquid droplets even at temperatures just below that of the drying chamber. In this case, it may be difficult to practically recover the pure AGIs from the spray dryer and, upon recovery, they may be physically unstable on exposure to an ambient atmosphere. Thus, in a preferred embodiment, the spray drying solution may comprise L-leucine or derivative thereof (e.g., iso-leucine) in an amount between about 5% and about 50% by weight relative to the AGI. Preferably, the solution may comprise L-leucine or derivative thereof in an amount between about 7% and about 30%, about 10% and about 25% by weight relative to the AGI. In a preferred embodiment, the solution may comprise L-leucine in an amount of about 20% by weight relative to the AGI.

Particles of the dry powder formulation comprising an adhesion modifier (e.g., L-leucine or magnesium stearate) will have at least a portion of the adhesion modifier located at the surface. The presence of adhesion modifiers on the surface of the particles may reduce the tendency of the particles to agglomerate. The relative proportion of adhesion modifier on the surface of the particles may be increased by high shear processing so as to further distribute the adhesion modifier over the particle surface. Shear processing may be carried out using any suitable method known in the art, for example, using a Eirich EL1 high shear blender or a Hosokawa Micron Cyclomix or AMS. In a preferred embodiment, the surface will comprise at least a 50% coverage by the adhesion modifier, more preferably more than 75% and most preferably more than 90%. It is believed that the concentration of adhesion modifier (e.g., L-leucine or magnesium stearate) at the surface acts to protect the dry active drug containing particles from agglomeration and the ingress of moisture. Assessment of adhesion modifier presence at the surface may be measured directly using a technique such as ToFSIMS (time of flight secondary ion mass spectrometry) or XPS (x-ray photoelectron spectroscopy). Alternatively it may be assessed by inverse phase gas chromatography. A preferred method to assess the presence of an adhesion modifier at the surface is an indirect approach via measurement of powder cohesion.

In some embodiments, the micronized particles of the dry powder comprising an AGI as disclosed herein are of a size suitable for aerosolisation and inhalation. For example, the micronized particles may have a physical size less than 15 μm, such as less than 10 μm, or less than 6 μm, or less than 5 μm, or less than 3 μm or less than 2 μm. The particles according to this embodiment will have a mass median aerodynamic diameter of less than 10 μm, but preferably less than 5 μm, or less than 3 μm.

Typically, in addition to the size equivalents discussed above, 90% of the particles by volume may have an aerodynamic diameter of less than 10 μm, less than 8 μm, or less than 6 μm or less than 5 μm or less than 3 μm.

In some embodiments, the spray dried formulations disclosed herein comprise or consist of an AGI and an adhesion modifier. Preferably, the spray dried formulation is a binary mixture of AGI and an adhesion modifier.

Where the micronized particles comprising an AGI as disclosed herein further include one or more pharmaceutically acceptable carriers, diluents or excipients, these pharmaceutically acceptable carriers, diluents or excipients may also be prepared with L-leucine or magnesium stearate or other adhesion modifiers present on their surfaces. Where these are larger carrier particles, typically larger than 20 microns median diameter, or larger than 40 microns, these may be coated with the adhesion modifier by high shear mixing. Larger carrier particles may comprise lactose, mannitol or other excipients know for use as carriers in inhalers. Some carriers may be as small as 10 microns, particularly when coated with an adhesion modifier, and may also be known in the art as fluidisation aids.

The powders disclosed herein may additionally be formulated by combination with any known carrier particles, or other additives excipients such as flavour, smell or organoleptic sense modifiers. Some improvement may also be achieved by pelletising the powder into soft pellets with improved powder flow, and appropriate selection of dry powder inhaler accordingly.

The particles of the dry powder formulation typically have a mass median aerodynamic diameter of less than 10 μm, more preferably less than 5 μm and most preferably less than 3 μm.

As used herein the term “aerodynamic diameter” (Dae) is defined as the diameter of an equivalent volume sphere of unit density with the same terminal settling velocity as the actual particle in question. Lung deposition of pharmaceutical powders is generally expressed in terms of particle's aerodynamic behaviour. The term “mass median aerodynamic diameter” (“MMAD”) is a statistical representation of the distribution of particle sizes graded according to aerodynamic diameter, defined herein as the median aerodynamic diameter expressed on a mass weighted basis, and is a widely accepted parameter used by aerosol scientists. The mass median aerodynamic diameter (MMAD) can be measured by a pharmacopeia impactor method as defined by the US Pharmacopeia, by using an Andersen cascade impactor or by Next Generation Impactor (NG1). In this respect, in order for the dry powder to be highly aerosolisable, the particles will generally have a mass median aerodynamic diameter of less than 10 but preferably less than 6 μm. preferably less than 5 μm, more preferably less than 3.5 μm or most preferably less than 2 μm.

The MMAD may also be approximated using a laser light scattering method, such as by using a Malvern Mastersizer 3000 instrument. While the Malvern Mastersizer 3000 measures mass median diameter (MMD), not MMAD, MMD can be used to approximate MMAD for comparative dispersion tests where the density of the particles is close to the density of the bulk material. The term “mass median diameter” (“MMD” or “D50”) refers to the particle diameter for which half of an aerosol mass is contained in particles having smaller diameters and the other half is contained in particles having larger diameters. Preferably, the particles of the dry powder formulations disclosed herein have an MMD of less than 10 μm, more preferably less than 5 μm and most preferably less than 3 μm. The MMD of particles of the dry powder formulations disclosed herein comprising an adhesion modifier may have a lower MMD than the corresponding formulation without an adhesion modifier. For example, the MMD of the dry powder formulations disclosed herein comprising an adhesion modifier may be 5%, 10%, 15%, 20%, 25%, 30%, 40%, 50% or more lower than the formulation without an adhesion modifier when measured at 0.2 Bar dispersion pressure. Preferably, the MMD of the dry powder formulations disclosed herein comprising an adhesion modifier it is at least 25% lower than the formulation without an adhesion modifier when measured at 0.2 Bar dispersion pressure.

The MMAD may be controlled, for example, by controlling the formation of the droplets during spray drying. For example, the choice of nozzle size, the velocity of the airflow and the drying process may be used to form droplets of a given size and having a narrow size distribution. Suitable methods for controlling the particle size and improving the fine particle dose of a spray dried powder are discussed in WO 2004/093848. The emitted dose (ED) is the total mass of the active agent emitted from the device following actuation. It does not include the material left on the internal or external surfaces of the device, or in the metering system including, for example, the capsule or blister. The ED is measured by collecting the total emitted mass from the device. It may be conducted in an apparatus frequently identified as a dose uniformity sampling apparatus (DUSA), and recovering this by a validated quantitative wet chemical assay (a gravimetric method is possible, but this is less precise). Alternatively, where an impactor or impinger is used, the ED is measured by combining the dose collected across all the stages of the respective impactor or impinger system.

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm, 2 μm or 1 μm, etc. The FPD is measured using an impactor or impinger, such as a twin stage impinger (TSI), multi-stage impinger (MSI). Andersen Cascade Impactor (ACI) or a Next Generation Impactor (NGI). When using a TS1, the FPD is generally taken at 6.4 μm as this impinger has only one cut point which is estimated at this value. Each impactor or impinger has a pre-determined aerodynamic particle size collection cut points for each stage. The FPD value is then obtained by interpretation of the stage-by-stage active agent recovery quantified by a validated quantitative wet chemical assay (a gravimetric method is possible, but this is less precise) where either a simple stage cut is used to determine FPD or a more complex mathematical interpolation of the stage-by-stage deposition is used. The fine particle fraction (FPF) is normally defined as the FPD divided by the ED and expressed as a percentage. Herein, the FPF of ED is referred to as FPF(ED) and is calculated as FPF(ED)=(FPD/ED)×100%. The fine particle fraction (FPF) may be defined as the FPD divided by the MD and expressed as a percentage. Herein, the FPF of MD is referred to as FPF(MD), and is calculated as FPF(MD)=(FPD/MD)×100%. The FPF(MD) can also be termed the ‘Dose Efficiency’ and is the amount of the dose of the pharmaceutical dry powder formulation which, upon being dispensed from the delivery device, is below a specified aerodynamic particle size.

In preferred embodiments, the dry powder formulations for use in the present invention have an FPF(ED) of at least about 40%, preferably between about 40 and 99%, more preferably between about 50 and 99%, even more preferably between about 60 and 99%. Further, in preferred embodiments, the dry powder formulations for use in the present invention preferably have an FPF(MD) of at least about 350%, preferably, between about 40 and 99%, more preferably between 50 and 99%.Preferably the adhesion modifier, such as L-leucine or magnesium stearate, represents between about 5 and 50% by weight of the dry ingredients of the powder formulations disclosed herein. More preferably, the dry power formulations according to the present invention may comprise an adhesion modifier in an amount of between about 10 and 40% by weight of the dry ingredients of the formulation. Preferably, the adhesion modifier, such as L-leucine or magnesium stearate represents between about 0.20 and 20% by weight of the active drug containing particles of the formulations disclosed herein. More preferably, the adhesion modifier comprises between 0.25 and 10% by weight of the active drug containing particles of the formulation.

Other suitable carriers, diluents, adjuvants and/or excipients for use in orally and/or nasally inhalable formulations will be known to those skilled in the art. In some embodiments, the compositions disclosed herein may further comprise an antioxidant, surfactant, co-solvent, adhesive, stabilizer, lubricant, humectant, osmolarity adjusting agent, pH modifying agent, sensory agent, preservative, penetration enhancer, chelating agent, sweetening agent, flavoring agent, taste masking agent, smell modifying agent, or colorant. Viscosity enhancers (e.g., glycerol, PVP or modified celluloses) may also be included to provide additional viscosity, moisture retention and/or a pleasant texture and odour for the formulation. Furthermore, some agents or components of the formulations disclosed herein may concurrently act, for example, as both a pH modifying agent and an osmolarity adjusting agent or as both sensory agent and a co-solvent. Where a given agent or component of an formulation as described herein is selected for a particular function, it is in no way taken to be limited to a single function only. It would be understood by a person skilled in the art that agents or components may additionally perform alternative or multiple functions.

In some embodiments, the AGIs and/or dry power formulations thereof as disclosed herein may be prepared as pharmaceutically acceptable emulsions, microemulsions, solutions, suspensions or similar such multiphasic composition or other art-known pharmaceutical dispersion. Preferably, the dosage form includes a carrier of isotonic saline and a pharmaceutically acceptable buffer. For aerosolisation, one or more propellants may also be included in the formulation. Suitable propellants will be known to those skilled in the art.

In some embodiments, formulations of the AGIs as disclosed herein may be prepared as aqueous solutions or suspensions for pulmonary administration. Where the formulations of the present invention are aqueous solutions or suspensions, the formulations may comprise water in an amount of greater than 50% by weight of the total composition, preferably greater than about 60% by weight of the total composition, more preferably greater than about 70% by weight of the total composition, even more preferably greater than about 80% by weight of the total composition. In still other embodiments, where the formulations disclosed herein are aqueous solutions or suspensions, the formulations may comprise water in the range of from about 80% to about 99% by weight of the total composition, more preferably from about 85% to about 98% by weight of the total composition.

In addition to the active ingredients, the liquid dosage forms disclosed herein may comprise inert diluents commonly used in the art such as, for example, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the liquid dosage may include adjuvants such as wetting agents, emulsifying and suspending agents, sweetening, flavoring, and perfuming agents. It is recognised that the additional inert diluents may also act as, for example, penetration enhancers, co-solvents or the like.

Thus, the present invention relates to inhalable composition comprising an α-glucosidase inhibitor and a pharmaceutically acceptable carrier, diluent, adjuvant or excipient. The compositions may be an orally inhalable composition, a nasally inhalable compositions, or both. In a preferred embodiment, the dry powder formulation is administered in the form of an aerosol.

Pharmaceutical compositions comprising an AGI for pulmonary administration as disclosed herein can be prepared by any method known in the art of pharmacology. In general, such preparatory methods include the steps of bringing the AGI into association with one or more carriers, diluents, adjuvants, excipients or other accessory ingredients (e.g., adhesion modifiers) and then, if necessary and/or desirable, shaping and/or packaging the product into a desired single- or multi-dose unit.

In certain embodiments, unit dosage compositions are those containing a daily dose or unit, daily sub-dose, as herein above described, or an appropriate fraction thereof, of the AGI. As used herein, a “unit dose” is discrete amount of the pharmaceutical composition comprising a predetermined amount of the active ingredient. The amount of the active ingredient is generally equal to the dosage of the active ingredient (i.e., the AGI) that would be administered to a subject and/or a convenient fraction of such a dosage such as, for example, one-half or one-third of such a dosage.

### Therapeutic Use

The AGIs disclosed herein, or compositions comprising the same, may be suitable for the treatment or prevention of a respiratory tract infection (RTI), including an upper RTI, a lower RTI, or a combination thereof. Furthermore, the AGIs disclosed herein may be suitable for the treatment or prevention of a viral RTI, a bacterial RTI, or a combination thereof. Thus, in some embodiments, the AGIs disclosed herein may be suitable for the simultaneous treatment or prevention of a viral RTI and a bacterial RTI.

It is also contemplated that, in addition to treating or preventing viral and/or bacterial RTIs, the AGIs disclosed herein may additionally treat or prevent any inflammatory sequelae to pulmonary infections known to trigger a local inflammatory response, for example, by modulation of one or more receptors associated with such inflammation. Thus, in some embodiments, the AGIs disclosed herein may be suitable for the simultaneous treatment or prevention of a viral RTI together with any associated inflammation, or a bacterial RTI together with any associated inflammation, or both a viral RTI and a bacterial RTI together with any inflammation associated with one or both of the viral RTI and the bacterial RTI. This may be particularly advantageous for improving treatment outcomes for pulmonary infections associated with an inflammatory response, such as SARS-CoV-2.

It is also contemplated that formulations comprising L-leucine as disclosed herein may also be useful for treating or preventing any inflammatory sequelae to pulmonary infections known to trigger a local inflammatory response, as L-leucine is known to metabolise in normal cells to produce β-hydroxy β-methylbutyrate (HMB), which has been shown to possess anti-inflammatory properties. Thus, in some embodiments, the AGI formulations disclosed herein may comprise HMB.

The AGIs as disclosed herein may be particularly suitable for the treatment of viral infections of the respiratory tract. Viruses generally lack their own glycosylation machinery and are therefore dependent on the host's machinery for viral replication. Virus replication often involves glycoproteins, for example as host cell surface receptors and/or viral attachment and fusion proteins. These glycoproteins undergo glycosylation, maturation and folding in the host endoplasmic reticulum (ER). AGIs, more particularly iminosugars, target the glucosidases that are important for entry into the glycoprotein folding cycle, interfering with the maturation of these essential glycoproteins and leading to misfolding (Alonzi et al. Biochem Soc Trans., 2017, 45(2), 571-582). Accordingly, AGIs are considered to provide broad spectrum antiviral activity. Advantageously, broad spectrum antiviral agents such as the AGIs disclosed herein may be useful for the treatment of viral co-infections of the respiratory tract, including where two or more viral infections occur simultaneously in the respiratory tract. Thus, in some embodiments, the AGIs disclosed herein may be useful for the treatment or prevention of one or more (e.g., two or three) viral respiratory tract infections.

Viral infections of the respiratory tract that may be treated by AGIs as disclosed herein include, but are not limited to, infections caused by an adenovirus, a coronavirus, an enterovirus, human metapneumovirus, an influenza virus, a parainfluenza virus, a respiratory syncytial virus or a rhinovirus, or any combination thereof. In a preferred embodiment, the AGIs disclosed herein may be suitable for the treatment of coronaviruses, e.g., Middle East respiratory syndrome (MERS-CoV), severe acute respiratory syndrome coronavirus 1 (SARS-CoV-1), severe acute respiratory syndrome coronavirus 2 (SARS-CoV-2), 229E, NL63, OC43 and HKU1. In a particularly preferred embodiment, the AGIs disclosed herein may be suitable for the treatment of SARS-CoV-2. The AGIs disclosed herein may be particularly useful for the treatment of viruses that currently cause significant disease in humans and/or are related to viruses that pose potential risks for originating future epidemics/pandemics. Such viruses may include the families: orthomyxoviridae (e.g., Influenza A H3N2, Influenza A H1N1 (eg pdm09) and Influenza B), paramyxoviridae (e.g., Nipah (NiV), Respiratory Syncytial Virus A and Respiratory Syncytial Virus B). coronaviridae (e.g., SARS, MERS, human coronavirus OC43) and picornaviridae (EV68, polio and enterovirus).

Suitable assays for evaluation of antiviral activity include, but are not limited to, virus inactivation assays, plaque reduction assays, cytopathic effect inhibition assays, binding and fusion assays (see, for example, Antiviral Methods and Protocols. Gong, E. Y., Ed. 2013. MIMB, volume 1030). Other suitable antiviral assays will be apparent to those skilled in the art. It has been demonstrated that AGIs as disclosed herein, have in vitro activity against a variety of viral RTIs, including severe acute respiratory syndrome (SARS), e.g., SARS-CoV-2, influenza, paramyxovirus and picornavirus. Many enveloped viruses may also be inhibited in vitro AGIs, including human immunodeficiency virus (HIV), Dengue, hepatitis C virus (HCV) and hepatitis B virus (HBV), coronaviruses (e.g., MERS, SARS and HCoV-229E, among others). Suitable assays for assessing activity against influenza and paramyxovirus are described by Watanabe and colleagues (MTT colorimetric assay system for the screening of anti-orthomyxo- and anti-paramyxoviral agents. J Virol Methods 1994, 48(2-3), 257-65), Bond and colleagues (The discovery of 1,2,3,9b-tetrahydro-5H-imidazo[2,1-a]isoindol-5-ones as a new class of respiratory syncytial virus (RSV) fusion inhibitors. Bioorganic & Medicinal Chemistry Letters. 2014, 25(4), 969-975) and MacDonald and colleagues (Potent and Long-Acting Dimeric Inhibitors of Influenza Virus Neuraminidase Are Effective at a Once-Weekly Dosing Regimen. Antimicrobial Agents And Chemotherapy, 2004, p. 4542-4549). A suitable assay for picornavirus is described by Feil and colleagues (An Orally Available 3-Ethoxybenzisoxazole Capsid Binder with Clinical Activity against Human Rhinovirus. ACS Med. Chem. Lett. 2012, 3(4), 303-307).

The present inventors have found that certain AGIs exhibit good in vitro activity (e.g., IC₅₀<100 μg/mL) against at least one strain of SARS-CoV-2 using the SARS-CoV-2 Virospot reduction assay (Viroclinics, Netherlands). In some embodiments, certain AGIs exhibit good in vitro activity against two, three or more strains of SARS-Cov-2. For example, glyset (miglitol), miglustat (NB-DNJ) and voglibose have been found to be active against at least one strain of SARS-CoV-2. IHVR-19029, NAP-DNJ and AMP-DNM and have been found to be active against at least three strain of SARS-CoV-2. In particular, NAP-DNJ and AMP-DNM have been shown to exhibit an IC₅₀<10 μg/mL and IC₉₀<40 μg/mL using the SARS—CoV-2 Virospot reduction assay. Given that NNDNJ, emiglitate and UV-4 are more lipophilic than miglustat and miglitol (glyset), it is expected that these compounds would also exhibit good in vitro activity against SARS-CoV-2. Further, while celgosivir exhibited an IC₅₀>200 μg/mL in the Virospot reduction assay, Rajasekharan et al. (Viruses, 2021, 13, 808) recently demonstrated that celgosivir was active against SARS-CoV-2 (EC₅₀ of 1±0.2 μM) using the human cell line Huh7 engineered to express the ACE2 receptor (Huh7-hACE2 cells). Miglustat was also found to be active in this system (EC₅₀ of 19.9±3.4 μM). In both cases, the number of viable nuclei increased upon treatment, a result of the protection from infection and lack of cytotoxicity up to the highest concentrations tested (500 and 200 μM, respectively). CC₅₀ was also measured by the Alamar blue assay with values exceeding 1000 μM for both drugs.

It is well known that the nature of the cell line can have an impact on the activity that is demonstrable in in vitro systems through, for example, effects of receptor expression, the differential expression of enzymes and other processes associated with prodrug activation, differences in expression and activity of host targets, such as glucosidases involved in post-translational modifications, among others. Therefore, the discovery of AGIs with SARS-CoV-2 activity can be challenging, as evidenced by the relative inactivity of the prodrug celgosovir in the Virospot reduction assay compared to the high activity reported by Rajasekharan et al. using a different assay and alternative cell lines. In this regard, it is believed that celgosivir is metabolised to castanospermine in the assay used by Rajasekharan et al., whereas the Virospot reduction assay lacks the necessary enzymic conversion. Therefore it is expected that both celgosivir and castanospermine would inhibit SARS-CoV-2 in vivo.

Thus, in a preferred embodiment, the present invention provides a method for treating or preventing a coronavirus infection. In particular, AGIs of Formula (ID) as described herein, or pharmaceutically acceptable salts thereof, may be particularly suitable for the treatment of a coronavirus infection, particularly SARS-CoV-2. Examples of AGIs of Formula (ID) that may be suitable for the treatment of SARS-CoV-2 include miglustat (NB-DNJ), glyset (miglitol), NNDNJ, UV-4, castanospermine, celgosivir, NAP-DNJ, IHVR-19029, AMP-DNM and emiglitate. Other AGIs may also be may be suitable for the treatment of a coronavirus infection, particularly SARS-CoV-2, such as volgibose.

Bacterial infections of the respiratory tract that may be treated by AGIs as disclosed herein include, but are not limited to infections caused by Streptococcus pneumonia, Staphylococcus aureus, Streptococcus pyogenes, Haemophilus influenza, Klebsiella pneumonia, Escherichia coli, Pseudomonas aeruginosa, Mycoplasma pneumonia, Legionella spp, Anaerobic bacteria, Mycobacterium tuberculosis, Chlamydia psittaci, Chlamydia trachomatis or Chlamydia pneumonia, or any combination thereof. The bacterial infection may be present in the upper RTI, lower RTI, or both. Suitable antibacterial assays will be apparent to those skilled in the art. For example, assays may be performed in accordance with the methods described in CLSI, Methods for Dilution Antimicrobial Susceptibility Tests for Bacteria that Grow Aerobically, Approved Standard, 9th ed., CLSI document M07-A9. Clinical and Laboratory Standards Institute, 950 West Valley Road, Suite 2500, Wayne, Pennsylvania 19087, USA, 2012.

The AGIs disclosed herein may be administered to a subject in need of treatment for an RTI, or they may be administered in a prophylactic sense. In particular, it is clear that the methods of the invention may be used prophylactically as well as for the alleviation of symptoms of a respiratory tract infection. References herein to “treatment” or the like may therefore include such prophylactic treatment, as well as therapeutic treatment of acute conditions or symptoms. Accordingly, in one or more embodiments, the present invention provides AGIs for use in the therapeutic treatment of respiratory tract infections. In other embodiments, the present invention provides AGIs for use in the prophylactic treatment of respiratory tract infections.

Accordingly, the present invention relates to a method of treating or preventing a respiratory tract infection comprising pulmonary administration of an effective amount of an AGI as disclosed herein to a subject.

The present invention also relates to use of an AGI in the manufacture of a medicament for treating or preventing a respiratory tract infection in a subject, wherein treating or preventing comprises pulmonary administration of the AGI to the subject.

The present invention further relates to an AGI for use in treating or preventing a respiratory tract infection in a subject by pulmonary administration.

The terms “treat”, “treating” or “treatment” with regard to a condition refers to alleviating or abrogating the cause and/or the effects of the condition. As used herein, the terms “treat”, “treatment” and “treating” refer to the reduction or amelioration of the progression, severity and/or duration of the condition, or the amelioration of one or more symptoms (e.g., one or more discernible symptoms) of the condition (i.e., “managing” without “curing” the condition), resulting from the administration of one or more therapies (e.g., one or more therapeutic agents such as a compound or composition as disclosed herein). In specific embodiments, the terms “treat”, “treatment” and “treating” refer to the amelioration of at least one measurable physical parameter of a condition described herein. In other embodiments the terms “treat”, “treatment” and “treating” refer to the inhibition of the progression of a condition described herein, either physically by, e.g., stabilization of a discernible symptom or physiologically by, e.g., stabilization of a physical parameter, or both.

The terms “preventing” and “prophylaxis” as used herein refer to administering a medicament beforehand to avert or forestall the appearance of one or more symptoms of a condition. The person of ordinary skill in the medical art recognises that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing” and “prevention” with regard to a condition refer to averting the cause, effects, symptoms or progression of a condition prior to the condition fully manifesting itself.

In some embodiments, the subject in need of treatment or prevention of respiratory tract infection is a mammal. The term “mammal” as used herein includes humans, primates, livestock animals (e.g., horses, cattle, sheep, pigs, donkeys), laboratory test animals (e.g., mice, rats, guinea pigs), companion animals (e.g., dogs, cats) and captive wild animals (e.g., kangaroos, deer, foxes). Preferably, the mammal is a human.

The AGIs disclosed herein are to be administered to the subject in need thereof in a treatment effective amount. In some embodiments, a treatment effective amount is a therapeutically effective amount or a prophylactically effective amount. The term “therapeutically effective amount” as used herein means an amount of AGI sufficient to treat or alleviate the symptoms associated with a respiratory tract infection. The therapeutically effective amount of the compound to be administered will be governed by such considerations, and is either, an incremental maximum tolerated dose, or the minimum amount, necessary to ameliorate, cure, or treat the condition or one or more of its symptoms. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Roughly, prophylactic measures are divided between primary prophylaxis (to prevent the development of a disease or symptom) and secondary prophylaxis (whereby the disease or symptom has already developed and the patient is protected against worsening of this process). Prophylaxis may include post-exposure prophylaxis (e.g., administering an effective amount of an AGI as disclosed herein to a subject known to have been exposed to a RTI).As used herein, the term “effective amount” relates to an amount of AGI which, when administered according to a desired dosing regimen, provides the desired therapeutic activity. For example, an effective amount of an AGI or stereoisomer thereof may be an amount sufficient to inhibit, slow, interrupt, halt, prevent or arrest viral or bacterial growth or replication. Suitable effective amounts may depend on the age, gender, weight and general health of the patient and can be determined by the attending physician. Suitable dosages lie within the range of about 0.1 ng per kg of body weight to 100 g per kg of body weight per dosage. The dosage may be in the range of 1 μg to 10 g per kg of body weight per dosage, such as is in the range of 1 mg to 1000 mg per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 200 mg per kg of body weight per dosage, such as up to 50 mg per kg body weight per dosage.

The terms “administer”, “administering” or “administration” in reference to a compound, composition or formulation disclosed herein means introducing the active agent (i.e., the AGI) into the system of the subject in need of treatment. When the active agent is provided in combination with one or more other active agents, “administration” and its variants are each understood to include concurrent and/or sequential introduction of the AGI and the other active agents.

In certain embodiments, an effective amount of an AGI for administration one or more times a day to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of an extract or compound per unit dosage form.

In certain embodiments, the inhalable compositions disclosed herein may be at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

The amount of AGI administered per dose or the total volume of composition administered will depend on such factors as the nature and severity of the symptoms, the age, weight, and general health of the patient, as well as the mode of administration. It is recognised that relative amounts of excipients, solvents, diluents, salts, thickening agents, sensory agents, buffers, and/or any additional ingredients in a pharmaceutical composition as disclosed herein may also depending upon the identity, size, and/or condition of the subject treated, as well as the mode of administration.

For example, in some embodiments, the dosage of AGI required to achieve a therapeutically equivalent effect may be greater for nasal inhalation dosage forms compared to oral inhalation dosage forms. The terms “therapeutic equivalence” or “therapeutically equivalent” as used herein refer to different compositions comprising the same active agent that produce the same clinical effect and safety profile and/or are pharmaceutical equivalents to one another.

The inhalable formulations disclosed herein may be administered in a single dose or a series of doses. Suitable dosage amounts and dosing regimens can be determined by the attending physician and may depend on the particular condition being treated, the severity of the condition as well as the general age, health and weight of the subject. It will be appreciated that dose ranges as described herein provide guidance for the administration of provided pharmaceutical compositions to an adult. The amount to be administered can be determined by a medical practitioner or person skilled in the art.

The formulations disclosed herein may be administered to a subject in need thereof by any suitable pulmonary delivery method. Suitable methods for pulmonary administration, including oral and nasal inhalation, would be well-known to a person skilled in the art.

Preferably, the inhalable compositions disclosed herein may be administered in the form of an aerosol. In particular, dry powder formulations as disclosed herein may be administered using equipment and techniques, known in the art. In this regard there are many inhalation devices described in the art for the purpose of allowing a patient to inhale a dry powder and this equipment may be used for the administration of the dry powders of the present invention.

Dry powder inhaler devices (DPIs) are well known in the art and there are a variety of different types. Generally, the dry powder is stored within the device and is extracted from the place of storage upon actuation, of the device, whereupon the powder is expelled from the device in the form of a plume of powder, which is to be inhaled by the subject. In most DPIs, the powder is stored in a unitary manner, for example in blisters or capsules containing a predetermined amount of the dry powder formulation. Some DPIs have a powder reservoir and doses of the powder are measured out within the device. These reservoir devices may be less favoured where the treatment is likely to be one or a small number of doses in an isolated treatment.

Dry powder inhalers may be passive or active. Passive inhalers are those whereby the powder is aerosolised using the air How drawn through the device by the patients inwards breath, and active devices are those whereby the powder is aerosolised by a separate source of energy, which may for example be a source of compressed gas such as the Nektar Exubera device or Vectura Aspirar device, or a form of mechanical energy such as vibration (such as the Microdose device) or impact.

The dry powder inhaler devices suitable for use in the present invention include “single dose” devices, for example the Rotahaler™, the Spinhaler™ and the Diskhaler™, in which individual doses of the powder composition are introduced into the device in, for example, single dose capsules or blisters. Devices may be presented as pre-metered for example with powder in a blister strip (as with the GSK Diskus device) where the pre-metered format comprises multiple doses) or where the patient inserts a pre-metered external dose form, such as a capsule containing the drug (for example the Boehringer Ingelheim Handihaler, or the Miat Monodose). Alternatively, the device may be a reservoir device, where the powder dose is metered within the device from a powder reservoir during patient handling (for example the Astra Turbuhaler). Any of these inhaler device types may be used.

The device may preferably be a single use device, or one that is designed for use with a small number of doses, and may be disposable. For example, the Twincer device, the Direct Haler device, the TwinCaps device or the Puff-haler. An advantage of these devices is their simplicity, small number of components and low cost. Preferably a device with fewer than 10 independent components is preferred. More preferably, 5 or fewer, most preferably 3 or fewer. There are a number of factors associated with the delivery devices which will affect the dosing efficiency achieved. Firstly, there is the extraction of the dose. Additionally, the dynamics of the powder plume generated will also affect dosing delivery. Preferably, the device will permit high emitted dose, and high efficiency de-agglomeration. High efficiency de-agglomeration is often associated with high levels of powder impaction on actuation. The device may have a low medium or high air flow resistance. A skilled person will appreciate that the compositions of the present invention can be administered with either passive or active inhaler devices.

Nasally inhalable compositions disclosed herein may be administered using any suitable methods known to a person skilled in the art. For example, the intranasal compositions disclosed herein may be administered as a spray or drop. Accordingly, suitable commercial packages containing the intranasal formulation can be in any spray container known in the art. In one or more embodiments, the formulations disclosed herein may be administered via a spray device or container. Spray devices may be single unit dose systems or multiple dose systems, for example comprising a bottle, a pump and/or an actuator. Such spray devices are available commercially. Suitable commercial spray devices include those available from Nemera, Aptar, Bespak and Becton-Dickinson. In still other embodiments, the formulations disclosed herein may be administered via an electrostatic spray device, such as described in U.S. Pat. No. 5,655,517. Other suitable means for administering formulations by nasal inhalation in accordance with the invention include via a dropper, a syringe, a squeeze bottle, and any other means known in the art for applying liquids nasally in an accurate and repeatable fashion.

The spray devices used to administer compositions as disclosed herein by nasal inhalation can range from single-use metered-dose spray devices, multiple-use metered dose nasal spray devices and are not limited to spraying the solutions into each naris but can be administered as a gentle liquid stream from a plunger, syringe or the like or as drops from a unit-dose or multi-dose squeeze bottle, or other means known in the art for administration by nasal inhalation in an accurate and repeatable fashion.

In one or more embodiments, a spray device suitable for use with the invention may typically deliver a volume of liquid in a single spray actuation in the range of from 0.01 to 0.15 mL. A typical dosing regimen for a nasal spray product may be in the range of one spray into a single nostril (naris) to two sprays into each nostril (naris). Repeat dosing of the same nostril (naris) may also be undertaken.

It is recognised that the dosing schedule for pulmonary administration, including a repeat dosing schedule, may be modified to obtain a desired pharmacokinetic profile. Further, the dosing schedule may be modified to achieve a rapid reduction in severity, preferably cessation, of symptoms associated with the RTI. In one or more embodiments, repeat dosing may occur every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, incremental increases in repeat dosing may be required to achieve a reduction in severity or cessation of symptoms of the RTI. For example, it may be necessary to increase each repeat dose by 25%, 50%, 75%, 100%, 150% or 200% in order to achieve a reduction in severity or cessation of symptoms of the RTI.

In certain embodiments, it is envisaged that the AGIs described herein may be administered to a subject in need thereof as a substitute or replacement for other traditional medication. In other embodiments, it is envisaged that AGIs disclosed herein be administered to a subject in need thereof as a supplement or adjunct to traditional medication. In still other embodiments, it is envisaged that AGIs disclosed herein may be administered to a subject in need thereof in the absence of adjunct therapy.

Replacing traditional medication for the treatment of respiratory tract infections with an AGI disclosed herein may be advantageous, particularly where the traditional medication is associated with one or more adverse effects.

### Combination Therapy

In other embodiments, the AGIs (including stereoisomers thereof) as disclosed herein may be administered to a subject in need thereof, together with one or more other medications for a discrete period of time, to address specific symptoms of RTIs. In still other embodiments, the person in need thereof may be treated with an AGI and one or more additional medications (administered sequentially or in combination) for the duration of the treatment period. Such combination therapy may be particularly useful, for example, where an additive or synergistic therapeutic effect is desired.

The AGIs disclosed herein may be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

The phrase “combination therapy” as used herein, is to be understood to refer to administration of an effective amount, using a first amount of, for example, an AGI or a pharmaceutically acceptable salt thereof as described herein, and a second amount of an additional suitable therapeutic agent.

When co-administered with other agent, an “effective amount” of the second agent will depend on the type of drug used. Suitable dosages are known for approved agents and can be adjusted by a person skilled in the art according to the condition of the subject, the type of condition(s) being treated and the amount of a compound, extract or composition being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

In certain embodiments, the AGIs disclosed herein (or a pharmaceutically acceptable salt thereof) and the additional therapeutic agent are each administered in an effective amount (i.e., each in an amount that would be therapeutically effective if administered alone). In other embodiments, the AGI and the additional therapeutic agent are each administered in an amount that alone does not provide a therapeutic effect (a sub-therapeutic dose). In yet other embodiments, the AGI can be administered in an effective amount, while the additional therapeutic agent is administered in a sub-therapeutic dose. In still other embodiments, the AGI can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a person in need thereof.

Co-administration encompasses administration of the AGI and one or more additional therapeutic agents in an essentially simultaneous manner, such as in a single pharmaceutical composition, for example, an aerosol or nasal spray having a fixed ratio of first and second amounts, or as discrete dosage forms. In addition, such co-administration also encompasses use of each compound in a sequential manner in either order. When co-administration involves the separate administration of a first amount of an AGI and a second amount of an additional therapeutic agent, they are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life, and kinetic profile.

In one or more embodiments where the AGI is administered with an additional therapeutic agent, the additional therapeutic agent may be any therapeutic agent that provides a desired treatment outcome. In particular, the additional therapeutic agent may be selected from known therapeutic agents for the treatment or prevention of respiratory tract infections, including one or more symptoms thereof.

The AGIs disclosed herein may be administered in combination with other anti-viral or anti-retroviral agents, anti-bacterial agents or other therapeutic agents suitable for use in the treatment of viral or bacterial infections, such as immunomodulators, immunostimulants, antibiotics, and the like. Non-limiting examples of suitable anti-viral or anti-retroviral agents may include chloroquine, hydroxychloroquine, tafenoquine, remdesivir, lopinavir, ritonavir, darunavir, favipiravir (favilavir), ribavirin, galidesivir, nitazoxanide, oseltamivir, zanamivir, umifenovir, tenofovir, niclosamide, rifampicin, mtricitabine, velpatasvir, ledipasvir, nelfinavir, darunavir, cobicistat, umifenovir, triazavirin, disulfiram, nitazoxanide, nafamostat, camostat mesylate, almitrine bismesylate, ivermectin, β-D-N4-hydroxycytidine, other iminosugars (such as those identified in PCT/US09/55658), poly(ADP-ribose) polymerase (PARP) inhibitor, stenoparib, fingolimod, colchicine, N4-hydroxycytidine, methylprednisone, oseltamivir, icatibant, perphanizine, viracept, emetine, homoharringtonine, aloxistatin, valrubicin, famotidine, almitrine, amprenavir, hesperidin, biorobin, cromolyn sodium, tocilzumab and sarilumab. Non-limiting examples of suitable immunomodulators and immunostimulants may include various interleukins, sidachomes, antibody preparations (such as monoclonal antibodies directed against key inflammatory cytokines or other aspects of the innate immune response, e.g., tocilizumab, sarilumab, baricitinib, imatinib, dasatinib, ruxolitinib, acalabrutinib), interferon-α and -β, cyclosporine, blood transfusions, and cell transfusions. Non-limiting examples of suitable antibiotics may include antifungal and antibacterial agents, examples of which are well known to those skilled in the art.

In some embodiments, the AGIs disclosed herein may be administered in combination with one or more anti-inflammatory agents. Non-limiting examples of suitable anti-inflammatory agents may include inhalable corticosteroids (such as budesonide, fluticasone, beclamethasone dipropionate, mometasone, ciclesonide), other corticosteroids (such as dexamethasone, prednisolone, prednisone, triamcinolone, bethamethasone, fludrocortisone, cortisone, hydrocortisone), non-steroidal anti-inflammatory agents (such as ibuprofen, asprin, naproxen, diclofenac, celecoxib), b-adrenergic receptor antagonists (such as nadolol), and AMPK activators (such as AICAR). Other suitable anti-inflammatory agents will be apparent to those skilled in the art.

Examples of other agents that may be co-administered with the AGIs disclosed herein include mucolytic and antithrombitic agents (such as heparin, acetyl cysteine, dipyridamole), other respiratory therapy drugs (such as beta agonists, long acting beta agonists, anticholinergic, anti-muscarinic and long acting anti-muscarinic agents), pulmonary fibrosis therapeutics (such as pirfenidone (Esbriet), nintedanib (Ofev), prednisone, mycophenolate, mofetil/mycophenolic acid, azathioprine, methotrexate, cyclophosphamide, rapamycin (sirolimus), and tacrolimus), pulmonary hypertension therapeutics, nicotine, terpines and related cannabinoid derivatives, zinc and nitric oxide generating materials (such as arginine). However, it will be appreciated that the AGIs may also be administered in combination with other agents useful for the treatment or prevention of respiratory tract infections, e.g., SARS-Cov-2, or other diseases and/or injuries associated with pulmonary inflammation.

Where an AGI is administered in combination with an additional therapeutic agent, the second agent may be administered in any “effective amount” which provides the desired therapeutic activity, as described above. Suitable dosage amounts and dosing regimens of the additional therapeutic agent can be determined by the attending physician and may depend on the particular condition being treated, the severity of the condition as well as the general age, health and weight of the subject. It will be appreciated that, unless otherwise specified, dose ranges as described herein provide guidance for the administration of provided pharmaceutical compositions to an adult. The amount to be administered to can be determined by a medical practitioner or person skilled in the art.

### Kits

The AGIs and formulations thereof as disclosed herein may be contained in a kit. The kit may include, for example, the AGI and an additional agent, each packaged or formulated individually, or packaged or formulated in combination. Thus, the AGI may be present in first container, and the kit can optionally include one or more agents in a second container. The container or containers are placed within a package, and the package can optionally include administration or dosage instructions. The kits disclosed herein may comprise the AGI in a form suitable for pulmonary administration (e.g., an inhaler or spray device and a container or packaging. The kits may optionally comprise instructions describing a method of using the pharmaceutical compositions in one or more of the methods described herein (e.g., for preventing or treating an RTI). The kit may optionally comprise a second pharmaceutical composition comprising one or more additional agents described herein for co-therapy use, a pharmaceutically acceptable carriers, diluents, adjuvants and/or excipients. The pharmaceutical composition comprising the AGI and the second pharmaceutical composition contained in the kit may be optionally combined in the same pharmaceutical composition.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples, which are intended for the purpose of illustration only and are not intended to limit the scope of the generality hereinbefore described.

### EXAMPLES

The in-vitro susceptibility of viruses to the α-glucosidase inhibitors described herein was assessed using a quantitative assay to measure virus replication in the absence or presence of graded concentrations of the test article (i.e., the AGI).

### Determination of Effective Concentration 50 (EC₅₀)

The percent cell protection achieved by the positive control and test articles in virus-infected cells was calculated by the formula of Pauwels et al. (Journal of Virological Methods, 1998, 20 (4), Pages 309-321) as shown below:

Percent cell protection=([ODt]virus−[ODc]virus/[ODc]mock−[ODc]virus)×100

where:
[ODt]virus=the optical density measured in a well examining the effect of a given concentration of test article or positive control on virus-infected cells;
[ODc]virus=the optical density measured in a well examining the effect of the negative control on virus infected cells; and
[ODc]mock=the optical density measured in a well examining the effect of the negative control on mock-infected cells.

EC₅₀ values were calculated from the percent cell protection results by non-linear regression analysis using the Hill (sigmoid Eₘₐₓ) formula as shown below:

$$y = {Min}_{y} + \frac{{Max}_{y} - {Min}_{y}}{1 + {( \frac{E {C}_{50}}{x} )}^{D}}$$

where:
x=test or control article concentration;
y=percent cell protection;
Min=minimum;
Max=maximum;
D=slope coefficient.

The 50% cytotoxic concentration (CC₅₀) is defined as the concentration of the test compound that reduces the absorbance of the mock infected cells by 50% of the control value. The CC₅₀ value was calculated as the ratio of [ODt]mock/[ODc]mock.

IDBS XLFit4 Excel Add-in (ID Business Solutions Inc., Alameda, CA) was used to perform the above calculations.

### Determination of Effective Concentration 50 (EC₅₀)

The percent cell protection achieved by the positive control and test articles in virus-infected cells was calculated by the formula of Pauwels et al. (Journal of Virological Methods, 1998, 20 (4), Pages 309-321) as shown below:

Percent cell protection=([ODt]virus−[ODc]virus/[ODc]mock−[ODc]virus)×100

where:
[ODt]virus=the optical density measured in a well examining the effect of a given concentration of test article or positive control on virus-infected cells;
[ODc]virus=the optical density measured in a well examining the effect of the negative control on virus infected cells; and
[ODc]mock=the optical density measured in a well examining the effect of the negative control on mock-infected cells.

EC₅₀ values were calculated from the percent cell protection results by non-linear regression analysis using the Hill (sigmoid Eₘₐₓ) formula as shown below:

$$y = {Min}_{y} + \frac{{Max}_{y} - {Min}_{y}}{1 + {( \frac{E {C}_{50}}{x} )}^{D}}$$

where:
x=test or control article concentration;
y=percent cell protection;
Min=minimum;
Max=maximum;
D=slope coefficient.

The 50% cytotoxic concentration (CC₅₀) is defined as the concentration of the test compound that reduces the absorbance of the mock infected cells by 50% of the control value. The CC₅₀ value was calculated as the ratio of [ODt]mock/[ODc]mock.

IDBS XLFit4 Excel Add-in (ID Business Solutions Inc., Alameda, CA) was used to perform the above calculations.

### Alpha-Glucosidase Inhibitors

The AGIs used in the following Examples are commercially available as follows:
Acarbose: Sigma-Aldrich/AK Scientific, Inc.
AMP-DNM: Cayman Chemical Company/ Santa Cruz Biotechnology, Inc.
Miglustat (NB-DNJ): Sigma-Aldrich/Biosynth Carbosynth Limited
Glyset (miglitol): Sigma-Aldrich/AK Scientific, Inc.
Emiglitate (BAY1248): LabNetwork/Medchemexpress
N-Non yldeoxynojirimycin (NNDNJ): Sigma-Aldrich/ Santa Cruz Biotechnology, Inc.
N-7-oxadecyldeoxynojirimycin (UV-4; SP 116): LabNetwork/Medchemexpress
Castanospermine: Sigma-Aldrich/Biosynth Carbosynth Limited
Celgosivir (6-O-butanoylcastanospermine): Sigma-Aldrich/Biosynth Carbosynth Limited
IHVR-19029: AbovChem LLC/Medchemexpress
Voglibose: Sigma-Aldrich/AK Scientific, Inc.

NAP-DNJ can be synthesised by one or more of the methods described in WO 2011028779, US 20070275998 A1 and Rawlings et al. (Synthesis and Biological Characterisation of Novel N-Alkyl-Deoxynojirimycin α-Glucosidase Inhibitors. ChemBioChem. 2009, 10(6), 1101-1105).

### Example 1

### Salt Formation of Miglustat and Miglitol

Miglustat L-Aspartate Salt

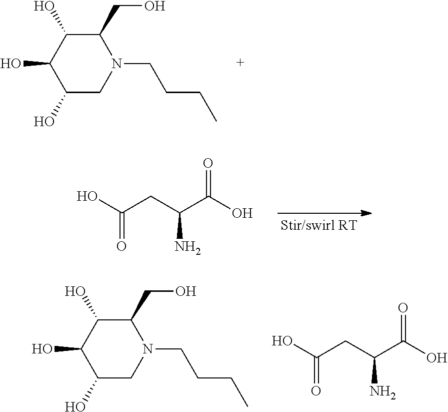

Miglustat (NB-DNJ) (5.00 g, 0.0228 mol, 1.0 eq), L-aspartic acid (3.03 g, 0.0228 mol, 1.0 eq), and distilled de-ionized water (100 mL) were combined in a 250 mL round bottom flask. The flask was swirled by hand for 5-10 minutes and most of the solid dissolved. Once all of the solid had dissolved the contents of the flask were frozen using liquid nitrogen and the flask placed on the freeze-drier and dried for 22 hours to give a colourless solid (8.60 g, quantitative yield). NMR:¹NMR (400 MHz, D₂O) δ 4.04 (dd, J=13.4, 1.7 Hz, 1H), 3.94 (dd, J=13.4, 3.0 Hz, 1H), 3.85 (dd, J=8.8, 3.8 Hz, 1H), 3.75 (ddd, J=11.4, 9.4, 4.9 Hz, 1H), 3.62 (dd, J=10.5, 9.4 Hz, 1H), 3.54 (dd, J=12.3, 5.0 Hz, 1H), 3.47 (t, J=9.4 Hz, 1H), 3.32 (td, J=12.3, 11.8, 5.3 Hz, 1H), 3.17 (ddd, J=12.9, 11.0, 5.6 Hz, 2H), 3.04 (t, J=11.9 Hz, 1H), 2.77 (dd, J=17.5, 3.8 Hz, 1H), 2.63 (dd, J=17.5, 8.8 Hz, 1H), 1.78-1.58 (m, 2H), 1.35 (hd, J=7.2, 2.5 Hz, 2H), 0.90 (t, J=7.4 Hz, 3H).

Miglustat L-Glutamate Salt

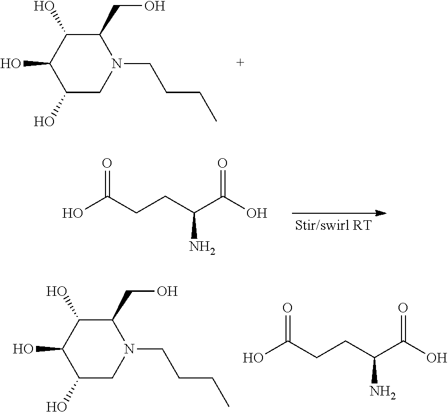

Miglustat (NB-DNJ) (5.00 g, 0.0228 mol, 1.0 eq), L-glutamic acid (3.35 g, 0.0228 mol, 1.0 eq), and distilled de-ionized water (100 mL) were combined in a 250 mL round bottom flask. The flask was swirled by hand for 5-10 minutes and most of the solid dissolved. Once all of the solid had dissolved the contents of the flask were frozen using liquid nitrogen and the flask placed on the freeze-drier and dried for 22 hours to give a colourless solid (9.10 g, quantitative yield). NMR: ¹NMR (400 MHz, D₂O) δ 4.04 (dd, J=13.4, 1.8 Hz, 1H), 3.95 (dd, J=13.4, 3.0 Hz, 1H), 3.81-3.67 (m, 2H), 3.62 (dd, J=10.5, 9.4 Hz, 1H), 3.54 (dd, J=12.3, 5.0 Hz, 1H), 3.47 (t, J=9.4 Hz, 1H), 3.32 (ddd, J=13.1, 11.3, 5.6 Hz, 1H), 3.23-3.10 (m, 2H), 3.04 (dd, J=12.3, 11.4 Hz, 1H), 2.44-2.24 (m, 2H), 2.16-1.94 (m, 2H), 1.68 (dddt, J=18.3, 14.5, 12.3, 6.3 Hz, 2H), 1.35 (ttd, J=14.3, 7.2, 2.6 Hz, 2H), 0.90 (t, J=7.4 Hz, 3H).

### Miglitol L-Aspartate Salt

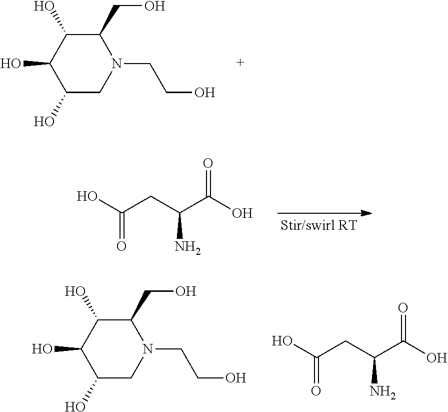

Glyset (miglitol) (5.00 g, 0.0241 mol, 1.0 eq), L-aspartic acid (3.21 g, 0.0241 mol, 1.0 eq), and distilled de-ionized water (100 ml) were combined in a 250 mL round bottom flask. The flask was swirled by hand for 5-10 minutes and most of the solid dissolved. Once all of the solid had dissolved the contents of the flask were frozen using liquid nitrogen and the flask placed on the freeze-drier and dried for 22 hours to give a colourless solid (9.01 g, quantitative yield). NMR: ¹NMR (400 MHz, D₂O) δ 4.09-3.89 (m, 4H), 3.86 (dd, J=8.6, 3.8 Hz, 1H), 3.79 (ddd, J=11.4, 9.4, 5.0 Hz, 1H), 3.70-3.61 (m, 2H), 3.55 (ddd, J=13.9, 7.0, 4.5 Hz, 1H), 3.48 (t, J=9.4 Hz, 1H), 3.29 (ddd, J=14.0, 5.3, 4.0 Hz, 1H), 3.21 (dt, J=10.5, 2.6 Hz, 1H), 3.07 (dd, J=12.3, 11.4 Hz, 1H), 2.79 (dd, J=17.6, 3.8 Hz, 1H), 2.65 (dd, J=17.6, 8.6 Hz, 1H).

### Miglitol L-Glutamate Salt

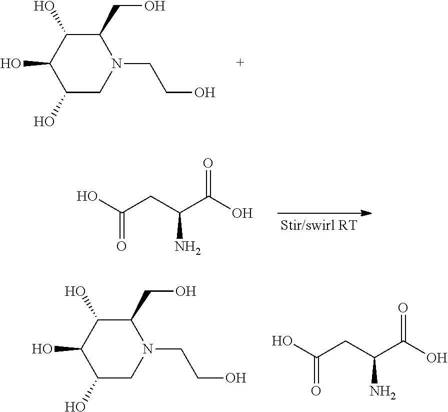

Glyset (miglitol) (5.00 g, 0.0241 mol, 1.0 eq), L-glutamic acid (3.55 g, 0.0241 mol, 1.0 eq), and distilled de-ionized water (100 ml) were combined in a 250 mL round bottom flask. The flask was swirled by hand for 5-10 minutes and most of the solid dissolved. Once all of the solid had dissolved the contents of the flask were frozen using liquid nitrogen and the flask placed on the freeze-drier and dried for 22 hours to give a colourless solid (9.18 g, quantitative yield). NMR: ¹NMR (400 MHz, D₂O) δ 4.07-3.96 (m, 2H), 3.95-3.85 (m, 2H), 3.77 (ddd, J=11.3, 9.4, 4.9 Hz, 1H), 3.70 (dd, J=7.1, 5.0 Hz, 1H), 3.66-3.58 (m, 2H), 3.57-3.42 (m, 2H), 3.26 (ddd, J=13.9, 5.4, 4.0 Hz, 1H), 3.16 (dt, J=10.5, 2.6 Hz, 1H), 3.08-2.98 (m, 1H), 2.41-2.24 (m, 2H), 2.15-1.94 (m, 2H).

### Example 2

### Spray Drying of Glyset (Miglitol)

### Sample Preparation

### Glyset (Miglitol) (Solution 2A):

Solution 2A was prepared by dissolving 10 g of glyset (miglitol) in 200 mL water with minor stirring to obtain complete dissolution.

Glyset (miglitol)/L-Leucine (Solution 2B):

Solution 2B was prepared by dissolving 8 g of glyset (miglitol) and 2 g of L-leucine in 200 mL water. The mixture was stirred for 5 minutes to obtain complete dissolution of the L-leucine.

### Spray Drying of the Solutions

An Across International SD-S15 Spray Dryer fitted with a 1.0 mm nozzle diameter was used in these examples. Any laboratory spray dryer such as a Buchi 290 or Procept Spray Dryer, of Niro SD Micro, would also be suitable for this process.

### Preparation of Spray Dryer

Prior to commencing the experiment, the Spray Dryer was first purged with approximately 50 mL of water. The fan was first turned on and set to 8, followed by the heating which was set to 190° C. Once the operating temperature of the machine was reached, the air pump was then turned on, followed by the peristaltic pump set to 30 and the pulse cleaning needle set to 8 seconds. The system was then allowed to reach a steady state.

### Experiment 2A—Spray Drying of Solution 2A

Upon completion of the preparation procedure, the water flow was then exchanged for the glyset (miglitol) solution (Solution 2A). Spray drying of the 200 mL volume took approximately 20 minutes under these conditions. Glyset (miglitol) proved to be extremely hygroscopic on collection in the cyclone. Rather than a dry powder, an initially dry deposit on the cyclone surface rapidly became a slurry accumulating about the cyclone chamber with very little material collected in the collection flask. No powder could be recovered.

### Experiment 2B—Spray Drying of Solution 2B

The experiment was then repeated under the same conditions to spray dry Solution 2B. This process also lasted approximately 20 minutes under these conditions. The glyset (miglitol)/L-leucine solution (Solution 2B) yielded a very different result under these conditions. A white particulate solid collected predominantly in the collection flask of the cyclone, but also formed deposits on the upper section of the cyclone. The resulting spray dried product was a very fine white powder.

The fine white powder product material could easily be recovered at ambient laboratory conditions, and was easily aerosolised (see Example 6), and exhibited relatively low-cohesive but a moderate adhesive character, such that it leaves a fine dust residue on any contact surfaces. A sample of the glyset (miglitol)/L-leucine spray dried product gave a bulk density of about 0.238 g/mL. Collection efficiency of this system was estimated at approximately 60%.

### SUMMARY

The glyset (miglitol)/L-leucine (Solution 2B) spray dried into a fine white and dusty product powder that aerosolised easily. The neat glyset (miglitol) product (Solution 2A) could not be recovered as a powder under these standard operating conditions due to its hygroscopicity.

### Example 3

### Spray Drying of Miglustat (NB-DNJ)

### Sample Preparation

### Miglustat (NB-DNJ) (Solution 3A)

Solution 3A was prepared by dissolving 5 g of miglustat (NB-DNJ) in 100 mL water with minor stirring for 5 minutes to ensure complete dissolution.

### Miglustat (NB-DNJ) (Solution 3B)

Solution 3B was prepared by dissolving 4 g of miglustat (NB-DNJ) and 1 g L-leucine (Sigma Aldrich) in 95 mL water with 5 mL of ethanol (added to facilitate dissolution of the L-leucine) with minor stirring for 10 minutes to obtain complete dissolution.

### Miglustat (NB-DNJ) (Solution 3C)

Solution 3C was prepared by dissolving 3 g of miglustat (NB-DNJ) and 0.75 g L-leucine (Sigma Aldrich) in 95 mL water with 5 mL of ethanol (added to facilitate dissolution of the L-leucine) with minor stirring for 10 minutes to obtain complete dissolution.

### Miglustat (NB-DNJ) Aspartate Salt (Solution 3D)

Solution 3D was prepared by dissolving 4 g of miglustat (NB-DNJ) aspartate salt as prepared in Example 1 and 1 g L-leucine (Sigma Aldrich) in 95 mL water with 5 mL of ethanol (added to facilitate dissolution of the L-leucine) with minor stirring for 10 minutes to obtain complete dissolution.

### Miglustat (NB-DNJ) Glutamate (Solution 3E)

Solution 3E was prepared by dissolving 3 g of miglustat (NB-DNJ) glutamate salt as prepared in Example 1 and 0.75 g L-leucine (Sigma Aldrich) in 85 mL water with 5 mL of ethanol (added to facilitate dissolution of the L-leucine) with minor stirring for 10 minutes to obtain complete dissolution.

### Spray Drying of the Solutions

A Buchi 290 laboratory Spray Dryer fitted with a 0.7 mm nozzle diameter was used in these examples. As with Example 2, any laboratory spray dryer such as an Across International SD-S15 or Procept Spray Dryer, or Niro SD Micro, would also be suitable for this process.

### Preparation of Spray Dryer

Prior to commencing the experiment, the Spray Dryer was first purged with approximately 20 mL of water. The aspirator was first turned on and set to 100%, followed by the heating which was set to the target Inlet Temperature. Once the target operating temperature of the machine was reached the atomising air pump was then turned on to an air flow of 45 L/hr, followed by the peristaltic pump set to 20%. The system was then allowed to reach a steady state, prior to switching the peristaltic pump to the sample to be spray dried.

### Experiment 3A—Spray Drying of Solution 3A

Preparation of the spray dryer was as described above, with the inlet temperature set to 180° C. (as the standard setting indicated for Buchi 290 operation), and similar to inlet temperature used in Example 2. The outlet temperature was recorded at around 125° C.

Upon completion of the preparation procedure, the water flow was then exchanged for the miglustat (NB-DNJ) solution (Solution 3(A)). Spray drying of the 100 mL volume took approximately 15 minutes under these conditions. White powder was observed to collect in the cyclone, but significant deposits of solid material were observed in lower drying chamber and at the inlet to the cyclone. On completion of the experiment, only about 300 mg of powder was recovered from the cyclone, giving recovery of approximately 6%. A large amount of off-white residue was found caked at the inlet of the cyclone which was solid rather than in powder form, which appeared to be the majority of the material by mass.

Experiment 3B—Spray Drying of Solution 3B

Preparation of the spray dryer was as described above, with inlet temperature set to 180° C. The outlet temperature was recorded at around 125° C.

Upon completion of the preparation procedure, the water flow was then exchanged for the miglustat (NB-DNJ) & L-leucine (Solution 3B).

Spray drying of the 100 mL volume took approximately 15 minutes under these conditions. An increased amount of white powder was observed to collect in the cyclone compared to Solution 3A, but again significant deposits of solid material were observed in lower drying chamber and at the inlet to the cyclone. On completion of the experiment, only about 1 g of powder was recovered from the cyclone, giving recovery of approximately 20%. A white residue was found caked at the inlet of the cyclone which was solid rather than in powder form.

It was concluded from observations of Experiments 3A and 3B, that the poor cyclone collection was likely due to low melting point of the powder produced, resulting in softening of the particles during collection, and a tendency to stick, and fuse at the lower drying chamber and cyclone inlet. Subsequent experiments were conducted at reduced inlet and outlet temperatures.

### Experiment 3C—Spray Drying of Solution 3C

Preparation of the spray dryer was as described above, with inlet temperature set to 135° C. The outlet temperature was recorded at around 80 to 85° C.

Upon completion of the preparation procedure, the water flow was then exchanged for the miglustat (NB-DNJ) & L-leucine (Solution 3C).

Spray drying of the 100 mL volume took approximately 15 minutes under these conditions. A reduced amount of solid material were observed in lower drying chamber and at the inlet to the cyclone. On completion of the experiment, about 1.1 g of powder was recovered from the cyclone pot and 0.87 g of powder recovered from the cyclone main body, giving recovery of approximately 30% in the cyclone pot and about 50% recovery from the cyclone.

### Experiment 3D—Spray Drying of Solution 3D

Preparation of the spray dryer was as described above, with an inlet temperature set to 135° C. The outlet temperature was recorded at around 80 to 85° C.

Upon completion of the preparation procedure, the water flow was then exchanged for the miglustat (NB-DNJ) aspartate salt & L-leucine (Solution (3D).

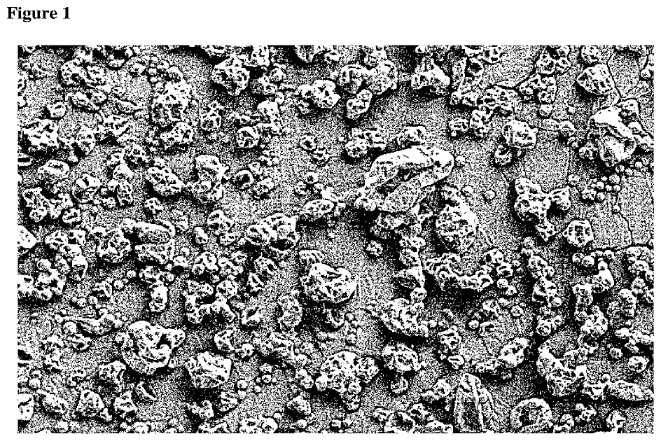

Spray drying of the 100 mL volume took approximately 15 minutes under these conditions. Spray drying appeared to be highly efficient, with most powder collecting in the cyclone pot, and little deposited in other parts of the cyclone or drying chamber. On completion of the experiment, about 3.2 g of powder was recovered from the cyclone pot, giving recovery of approximately 65% in the cyclone pot. The powder had a pure white and highly aerosolisable behaviour, generating large aerosolised clouds on agitation. SEM imaging of the spray dried salt showed the unagglomerated nature of particles and distinct wrinkled morphology to aid dispersion (FIG. 1).

### Experiment 3E—Spray Drying of Solution 3E

Preparation of the spray dryer was as described above, with inlet temperature set to 135° C. The outlet temperature was recorded at around 80 to 85° C.

Upon completion of the preparation procedure, the water flow was then exchanged for the miglustat (NB-DNJ) glutamate salt & L-leucine (Solution 3E).

Spray drying of the 90 mL volume took approximately 15 minutes under these conditions. Spray drying appeared to be efficient, with most powder collecting in the cyclone pot, and little deposited in other parts of the cyclone or drying chamber. On completion of the experiment, about 2.5 g of powder was recovered from the cyclone pot, giving recovery of approximately 67% in the cyclone pot. As with Experiment 3D, this powder had a pure white and aerosolisable behaviour on recovery. However, unlike Experiment 3D, the power produced from Experiment 3E was noted to be hygroscopic on recovery, picking up water and rapidly becoming more cohesive, sticking to contacted surfaces after a few minutes of exposure to ambient air with relative humidity of 50 to 60%.

### Example 4

### Micronization and Co-Micronization

A Sturtevant Micronizer Fluid Energy 4 inch Jet mill was uses to micronize a series of glyset (miglitol) examples to demonstrate the optimisation required for providing powders of the invention. Glyset (miglitol) was used as a demonstration material, and its physical properties mean it will be similar to other aminoglycosides of the invention.

The jet mill was set up with a feed pressure of approximately 4 Bar and a grinding pressure of approximately 7 Bar. Optimisation to ensure efficient feeding and milling is required in each case. The milled powder is collected into the cyclone and bag filter located above the cyclone exit.

### Experiment 4A—Micronization of Glyset (Miglitol)

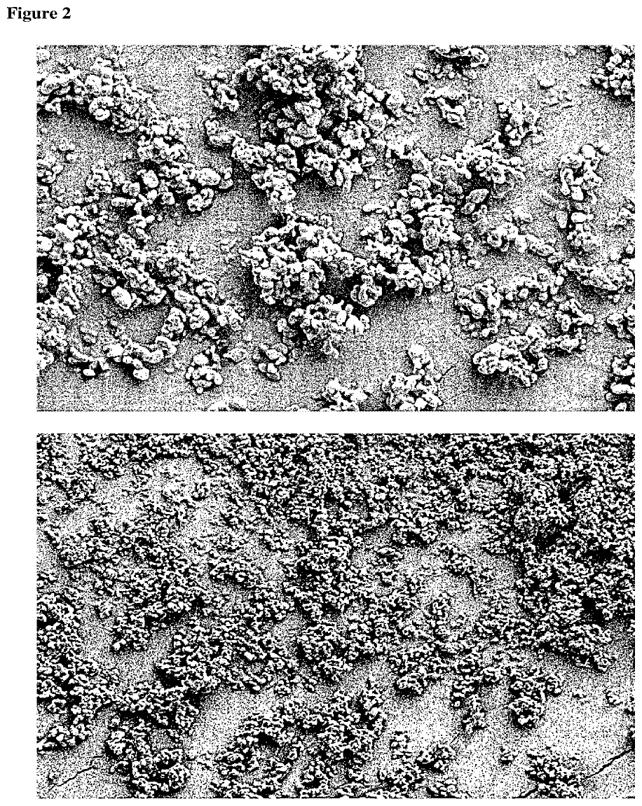

5.11 g of glyset (miglitol) was weighed out and manually fed into the mill at a constant rate, of approximately 2 g per minute. On completion of the milling the processed powder was recovered from the system by agitating the filter bag above the cyclone. It was noted that the amount of powder recoverable from this system was low. Approximately 0.37 g of milled powder was recovered representing around 7%. The residual powder appeared to be stuck in the bag filter media, attributed to its cohesivity. SEM imaging of the powder showed highly agglomerated clusters of micronized particles (FIG. 2).

Experiment 4B—Co-Micronization of Glyset (miglitol)+2% Magnesium Stearate

5.14 g of glyset (miglitol) was weighed out and combined with 0.10 g of magnesium stearate. The mix was hand blended by spatula for approximately 2 minutes. This mix was manually fed into the mill at a constant rate, of approximately 2 g per minute. On completion of the milling the processed powder was recovered from the system by agitating the filter bag above the cyclone. Approximately 2.51 g of the milled powder was recovered, representing around 48%. This powder was notably less cohesive than the powder from Experiment 4A.

### Experiment 4C—Co-Micronization of Glyset (Miglitol)+5% Magnesium Stearate

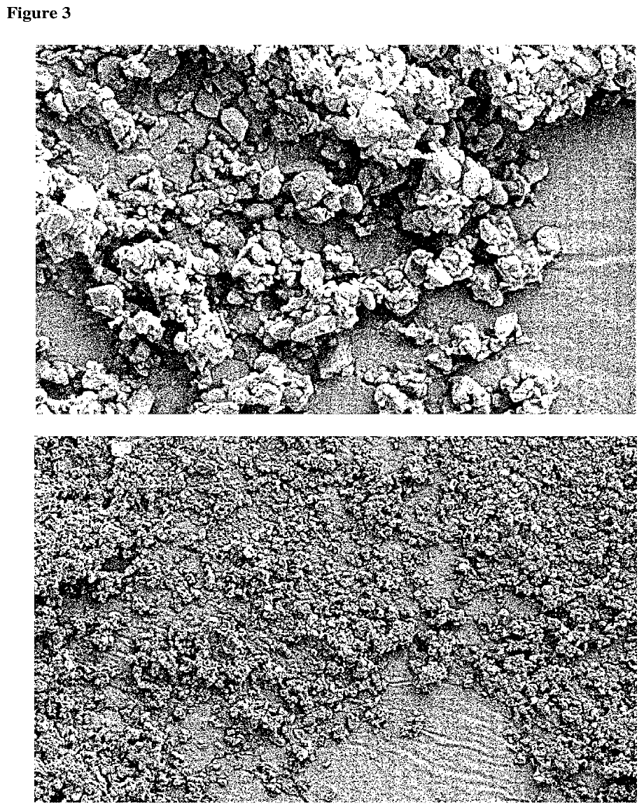

5.06 g of glyset (miglitol) was weighed out and combined with 0.25 g of magnesium stearate. The mix was hand blended by spatula for approximately 2 minutes. This mix was manually fed into the mill at a constant rate, of approximately 2 g per minute. On completion of the milling the processed powder was recovered from the system by agitating the filter bag above the cyclone. Approximately 3.1 g of the milled powder was recovered representing around 58%. This powder was also notably less cohesive than the powder from Experiment 4A. SEM imaging of the powder showed a mix of agglomerated clusters and more dispersed primary micronized particles (FIG. 3).

### SUMMARY

It was clear that co-micronization as described here provided a surprisingly enhanced process, allowing much easier and greater recovery of micronized powder in comparison to micronization of the aminoglycoside alone.

### Example 5

### High Energy Co-Mixing of Co-Micronization Powder (Mechanofusion)

Samples from the co-micronization of Experiments 4B and 4C were combined to provide sufficient powder mass to process in a Hosokawa AMS Mini Nobilta powder processor, with a 1 mm gap processing stator. A resulting sample of 4.6 g comprising co-micronized miglustat (NB-DNJ) with 3.7% w/w magnesium stearate was loaded into the Nobilta processor.

The powder was first pre-blended by slowly increasing speed up to 1000 rpm over 1 minute, and holding at 1000 rpm for 1 minute before stopping. This was intended to ensure a good mix in the powders.

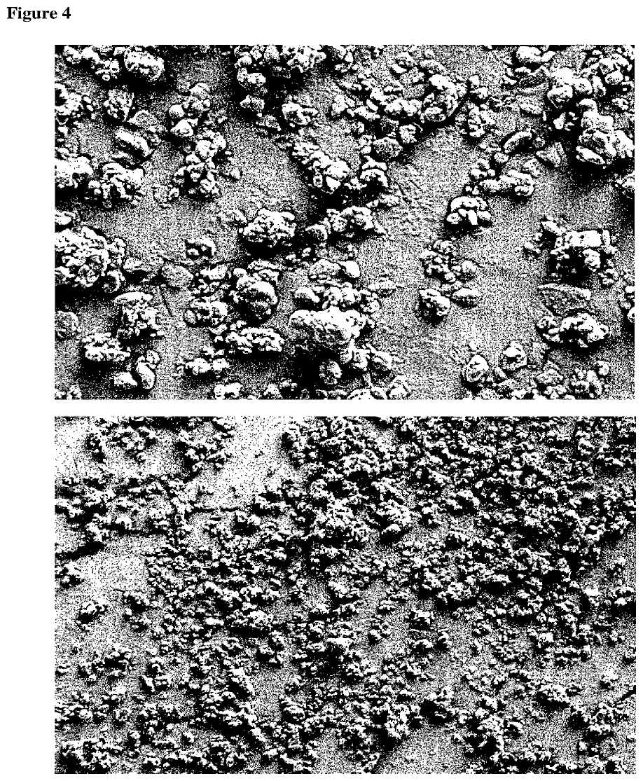

The powder was then processed by slowly increasing speed up to 3000 rpm over 1 minute, and holding at about 3000 rpm for 5 minutes. Power readings fell from 50 W to 44 W over this time, indicating reduced resistance in the processor, which may reflect reduced cohesion in the powder. SEM imaging showed far more dispersed primary micronized particles (FIG. 4) than was observed for the powders of Experiments 4A, 4B and 4C.

The poured bulk density of the powder was measured at 3 g/cc, which is a high density for a micronized pharmaceutical powder.

Higher densities are advantageous as this allows greater dosing level in a given volume for a dry powder inhaler. This would allow a size 3 HPMC capsule, such as used in a Miat Monohaler or Novartis Breezhaler, with volume 0.3 cc, to hold an untapped dose up to 90 mg, and would allow a size 2 HPMC capsule, such as used in a PH&T Turbospin or Novartis Podhaler, with volume 0.37 cc, to hold an untapped dose up to 110 mg.

This compares with a poured bulk density of the powder from Experiment 3D, which was measured at 0.19 g/cc.

### Example 6

### Assessment of Processed Micro-Powder Aerosolisation

To compare the relative aerosolisation of the formulated powders, a method originally developed by Harris and Morton, (“Powder Dispersibility: A Screening Method for Dry Powder Inhaler Development”, Proceedings of Aerosol Society Drug Delivery to the Lungs XIV, London, UK, 2003) was adapted to evaluate the powders from Examples herein.

In these tests, approximately 30 mg of powder from each sample was fed into a Malvern Mastersizer 3000 dry dispersion unit, using pre-determined parameters (i.e. values for powder refractive index). Dispersion was performed at four applied dispersion air pressures (0.1 Bar—being the lowest value possible in the system, 0.2 Bar, 0.5 Bar and 1 Bar). Particle size distribution data were derived, and as per previous studies with this technique, the dispersion was represented by the Mass Median Diameter (D50). The MMD was considered a valid approximation for MMAD due to the absence of carrier particles.

Tests were conducted on powders from Experiments 4A and 4C to evaluate the effect of co-micronizing with magnesium stearate on powder dispersibility, and then on powders from Examples 3 and 5. Only enough powder was available from Experiment 3A for 1 dispersion, which was dispersed at 0.5 Bar as a discerning pressure.

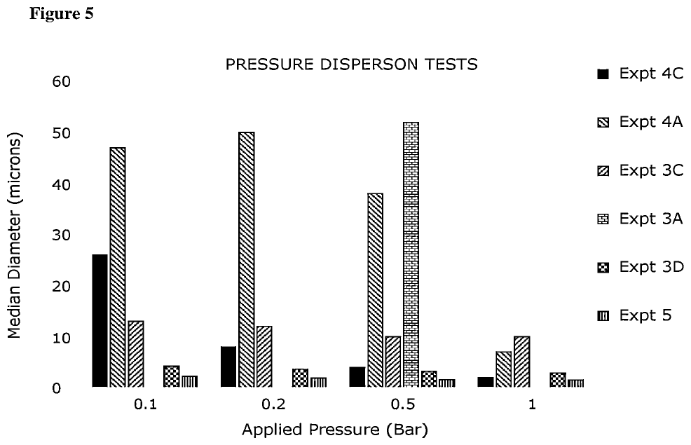

The results are presented in FIG. 5 and Tables 1 and 2 below, wherein the median diameter (D50) is the value at with the portions of particles with diameters above and below than this value are 50% and D90 is the value at which the portion of particles with diameters below this value is 90%.

TABLE 1
Pressure dispersion tests Medium Diameter (D50)

| Pressure | Median Diameter (microns) | | | | | |
|---|---|---|---|---|---|---|
| (Bar) | Expt 4C | Expt 4A | Expt 3C | Expt 3A | Expt 3D | Expt 5 |
| 0.1 | 26 | 47 | 13 | | 4.2 | 2.2 |
| 0.2 | 8 | 50 | 12 | | 3.6 | 1.9 |
| 0.5 | 4 | 38 | 10 | 52 | 3.2 | 1.6 |
| 1 | 2 | 7 | 10 | | 2.9 | 1.5 |

TABLE 2
Pressure dispersion tests D90

| | |
|---|---|
| Pressure | Diameter, D90 (microns) |

| (Bar) | Expt 4C | Expt 4A | Expt 3C | Expt 3D | Expt 5 |
|---|---|---|---|---|---|
| 0.1 | 1210 | 1230 | 133 | 30 | 13.1 |
| 0.2 | 124 | 176 | 85 | 8.8 | 9.8 |
| 0.5 | 73 | 1430 | 70 | 7 | 7 |
| 1 | 37 | 56 | | 6 | 3.9 |

It is clear from these pressure dispersion tests that the formulated powders exhibit very different comparative aerosolisation behaviours. The Experiment 3C powder, miglustat (NB-DNJ) co-spray dried with 20% w/w L-leucine at an inlet temperature of 135° C., provided near complete dispersion at the lowest pressure of 0.1 Bar, and was well dispersed at all pressures. It should be noted the Mass Median Diameter determined by Malvern Mastersizer is derived by light scatter, so is a geometric diameter rather than an aerodynamic diameter. It will be understood by those skilled in the art that for spray dried powders, the particle density is often reduced compared to micronized powders, and therefore these measurements appear larger than the effective aerodynamic diameter. In comparison, the only dispersion measurement of the spray dried miglustat (NB-DNJ) without L-leucine, from Experiment 3A, was very poorly dispersed at 0.5 Bar.

The Experiment 4C powder glyset (miglitol) co-micronized with 5% w/w magnesium stearate, provides a moderate dispersion at 0.1 Bar, and good dispersion at 0.2 Bar and full dispersion at 0.5 bar and 1 Bar. In comparison, the Experiment 4A powder, micronized glyset (miglitol), provides a poor dispersion at all pressures up to 0.5 Bar, with good dispersion only at 1 Bar. Experiment 5 shows that relative aerosolisation is further improved by co-jet milling followed by a high shear processing to improve coverage of the adhesion modifier on the particle surface.

These dispersion tests clearly illustrate the technical demands of establishing a suitable formulation approach for drug powders into a drug rich dispersible form to address the challenge of high dose delivery required for the invention. The development of high dose delivery formulation is a major challenge and is a specific case in each new class of drug molecules.

### Example 7

### SARS-CoV-2 Assay

SARS-CoV-2 testing was carried out by the independent contract research organization Viroclinics, Netherlands, using the SARS—CoV-2 Virospot reduction assay. This is a high throughput assay to determine the IC₅₀/IC₉₀ (also termed EC₅₀/EC₉₀) of putative antiviral compounds as compared to other assay formats (such as traditional plaque count assays), and improved traceability from raw data to results. The assay measures drug or antibody-mediated reduction of virus propagation in cell monolayers, by virus-specific immunostaining and automated imaging of infected cells. Automated counting of the number of infected foci (i.e., “spots”) provides the raw data for IC₅₀/IC₉₀ calculations.

A serial dilution series of the test article is mixed with a fixed amount of virus and the virus/inhibitor is added to Vero E6 cells. Virus control wells without inhibitor and cell controls without drug and virus are included. Viral propagation is measured after 16-24 h incubation by TrueBlue immunostaining of infected cells and automated counting of viral “spots” (corresponding to area of viral growth and protein expression) and the IC₅₀ value is calculated using 4 replicates.

### Method

Adherent cells were seeded in multi-well plates. Cell cultures were inoculated with a standardized amount of virus, in the absence and presence of serial compound dilutions, followed by 18-24 hours of incubation and an appropriate virus detection method (Immunostaining and count spots using a CTL Immunospot Image Analyser).

Virus signals were detected in presence of each compound concentration determine the 50% inhibitory concentration (IC₅₀) and the 90% inhibitory concentration (IC₉₀). The IC₅₀ and IC₉₀ values were calculated from the virus signals using the method described by Zielinska et al. (Virol J. 2005, 2(84)).

Remdesivir was used as a positive control.

Although cytotoxicity was not directly measured, the assay required intact cell monolayers to deliver a result. Severe cytotoxicity would be evident upon visual inspection and any deleterious effect of compound upon cell health is likely to negatively impact the formation of “spots”. This was not reported.

The activity of various AGIs against three different strains of SARS-CoV-2 was measured as shown in Table 3.

TABLE 3
Activity of AGIs against SARS-CoV-2

| | | IC₅₀ | IC₉₀ |
|---|---|---|---|
| Name | Virus | (μg/mL) | (μg/mL) |
| Acarbose | SARS-Cov-2 Germany/BavPat1/2020 | >200 | >200 |
| Glyset (Miglitol) | SARS-Cov-2 Germany/BavPat1/2020 | >200 | >200 |
| Miglustat (NB-DNJ) | SARS-Cov-2 Germany/BavPat1/2020 | >200 | >200 |
| Miglustat (NB-DNJ) | SARS-Cov-2 Germany/BavPat1/2020 | >200 | >200 |
| Voglibose | SARS-Cov-2 Germany/BavPat1/2020 | >200 | >200 |
| Celgosivir (6-O- | SARS-Cov-2 Germany/BavPat1/2020 | >200 | >200 |
| butanoylcastanospermine) | | | |
| AMP-DNM | SARS-Cov-2 Germany/BavPat1/2020 | 8.3 | 28.6 |
| IHVR-19029 | SARS-Cov-2 Germany/BavPat1/2020 | 48.5 | >200 |
| NAP-DNJ | SARS-Cov-2 Germany/BavPat1/2020 | 1.4 | 10.2 |
| Acarbose | SARS-Cov-2 SA (B.1.351) | >200 | >200 |
| Glyset (Miglitol) | SARS-Cov-2 SA (B.1.351) | 90.0 | >200 |
| Miglustat (NB-DNJ) | SARS-Cov-2 SA (B.1.351) | 19.2 | >200 |
| Miglustat (NB-DNJ) | SARS-Cov-2 SA (B.1.351) | 15.0 | >200 |
| Voglibose | SARS-Cov-2 SA (B.1.351) | 91.2 | >200 |
| Celgosivir (6-O- | SARS-Cov-2 SA (B.1.351) | >200 | >200 |
| butanoylcastanospermine) | | | |
| AMP-DNM | SARS-Cov-2 SA (B.1.351) | 2.7 | 24.8 |
| IHVR-19029 | SARS-Cov-2 SA (B.1.351) | 5.5 | >200 |
| NAP-DNJ | SARS-Cov-2 SA (B.1.351) | 0.1 | 5.7 |
| Acarbose | SARS-Cov-2 UK (B.1.1.7) | >200 | >200 |
| Glyset (Miglitol) | SARS-Cov-2 UK (B.1.1.7) | >200 | >200 |
| Miglustat (NB-DNJ) | SARS-Cov-2 UK (B.1.1.7) | >200 | >200 |
| Miglustat (NB-DNJ) | SARS-Cov-2 UK (B.1.1.7) | >200 | >200 |
| Voglibose | SARS-Cov-2 UK (B.1.1.7) | >200 | >200 |
| Celgosivir (6-O- | SARS-Cov-2 UK (B.1.1.7) | >200 | >200 |
| butanoylcastanospermine) | | | |
| AMP-DNM | SARS-Cov-2 UK (B.1.1.7) | 7.7 | 34.4 |
| IHVR-19029 | SARS-Cov-2 UK (B.1.1.7) | 58.5 | >200 |
| NAP-DNJ | SARS-Cov-2 UK (B.1.1.7) | 1.7 | 7.9 |

### Example 8

### Alternative SARS-CoV-2 Antiviral Assay

An alternative method for assessing activity against SARS-CoV-2 is as follows.

SARS-CoV-2 hCoV-19/Australia/VIC_{01/2020} may be grown in African Green Monkey Kidney (Vero) cells (ATCC-CCL81) using virus growth media, comprising Minimal Essential Medium without L-glutamine supplemented with 1% (w/v) L-glutamine 1.0 μg/mL of TPCK-Trypsin, 0.2% BSA, 1× Pen/Strep, and 1% Insulin Transferrin Selenium (ITS).

Vero cells may be seeded into 96-well plates at 2×104 cells/well in 100 μL seeding media (Minimal Essential Medium supplemented with 1% (w/v) L-glutamine, 1% ITS, 1× Pen/Strep, 0.2% BSA). Plates may be incubated overnight at 37° C., 5% CO₂.

Stock solutions of antiviral agents may be prepared fresh on the day of testing, vortexed and visually inspected to confirm complete dissolution. A positive control compound, remdesivir, may be prepared as a 10 mM stock in DMSO and stored at −20° C.

A DMSO dilution series of antiviral agents may be performed by addition of 10 μL (5 mg/mL) to rows A and B, column 2 of a v-bottom skirted PCR plate. A volume of 15 μL DMSO may be added to rows A and B, columns 3-11. Antiviral agents may be serially diluted 1:3 by transfer of 5 μL compound from column 2 to column 3, column 3 to column 4 and continued to column 10 and then discarded.

An intermediate dilution series in virus growth media (Minimal Essential Medium supplemented with 1% (w/v) L-glutamine, 1% ITS, 0.2% BSA, 1 μg/mL TPCK-Trypsin, 1× Pen/Strep) may be generated by transfer of 4 μL of compound from rows A and B, columns 2-11 into 496 μL virus growth media.

A 50 μL volume from each compound intermediate dilution series may be added to the rows B-G of an assay plate.

A 50 μL volume of SARS-CoV-2 diluted in virus growth media to generate a multiplicity of infection (moi) of 0.05, may be added to five 96-well plates. This moi was previously determined to provide 100% CPE in 4 days. Virus may be added to rows B, C and D to assess antiviral activity and virus growth media without virus added to rows E, F and G to assess cytotoxicity. Plates may be incubated at 37° C., 5% CO₂ for 4 days prior to staining with MTT (3-(4,5-dimethylthiazol-2-yl)-2,5-diphenyltetrazolium bromide).

After incubation for four days, viable cells may be determined by staining with MTT. A 100 μL volume of a 3mg/mL solution of MTT may be added to plates and incubated for 4 hours at 37° C. in a 5% CO2 incubator. Wells may be aspirated to dryness using a multichannel manifold attached to a vacuum chamber and formazan crystals solubilised by the addition of 200 μL 100% 2-propanol at room temperature for 30 minutes. Absorbance may be measured at 540-650 nm on a plate reader.

### REFERENCES

Alonzi D S, Scott K A, Dwek R A, Zitzmann N. Iminosugar antivirals: the therapeutic sweet spot. Biochem Soc Trans. 2017, 45(2), 571-582. doi:10.1042/BST20160182

Hickey A J. Advanced Drug Delivery Reviews, Pages Ahead of Print, 2020, doi: 10.1016/j.addr.2020.07.006

Pan Y T, Hori H, Saul R, Sanford B A, Molyneux R J, Elbein A D. Castanospermine inhibits the processing of the oligosaccharide portion of the influenza viral hemagglutinin. Biochemistry. 1983, 22(16), 3975-3984. doi:10.1021/bi00285a038

Rajasekharan S, Bonotto R M, Kazungu Y, Alves L N, Poggianella M, Orellana P M, Skoko N, Polez S, Marcello A. Repurposing of Miglustat to inhibit the coronavirus Severe Acquired Respiratory Syndrome SARS-CoV-2. 2020, bioRxiv. doi: 10.1101/2020.05.18.101691

Romero P A, Datema R, Schwarz R T. N-methyl-l-deoxynojirimycin, a novel inhibitor of glycoprotein processing, and its effect on fowl plague virus maturation. Virology. 1983, 130(1), 238-242. doi:10.1016/0042-6822(83)90133-2

1. A method for treating or preventing a viral respiratory tract infection in a subject comprising pulmonary administration of a therapeutically effective amount of an α-glucosidase inhibitor to the subject, wherein the α-glucosidase inhibitor is a compound of Formula (ID): 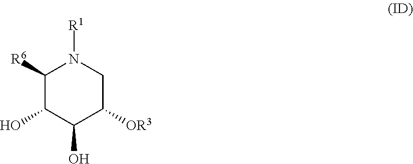
or a pharmaceutically acceptable salt thereof,
wherein:
R¹ is Ci-ioalkyl optionally substituted with one or more R^{X} groups, or LR⁹;
L is a divalent linker group selected from C₁₋₁₀alkyl-O—, or C₁₋₁₀alkyl-NR⁷-;
R⁹ is selected from C₁₋₄alkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl, or C₁-9heteroaryl group optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁₋₉heteroaryl optionally substituted with one or more R^{X} groups;
R³ is H or C(O)C₁₋₆alkyl;
R⁶ is CH₂—OH;
R⁷ and R⁸ are independently selected from H, C₁₋₄alkyl, C₃₋₆cycloalkyl, or C(O)NH-C₁₋₄alkyl;
or wherein R¹ and R⁶ together with the atoms to which they are attached form a 5-membered heterocycloalkyl ring substituted with a hydroxyl group; and
each R^{X} is independently selected from hydroxy, halo, nitro, azido, C₃₋₁₀cycloalkyl, C₁₋₄alkoxy and CO(O)C₁₋₄alkyl.

2. The method of claim 1 wherein the respiratory tract infection is a viral infection is a coronavirus infection.

3. The method of claim 2, wherein the coronavirus infection is SARS-CoV-2.

4. The method of claim 1, wherein R¹ is C₁₋₆alkyl optionally substituted with one or more R^{X} groups or LR⁹.

5. The method of claim 1, wherein L is a divalent linker group selected from C₁₋₆alkyl-O—, or C₁₋₆alkyl-NR⁷—.

6. The method of claim 1, wherein each R^{X} is independently selected from hydroxy, halo, nitro, azido and C₃₋₁₀cycloalkyl.

7. The method of claim 1, wherein 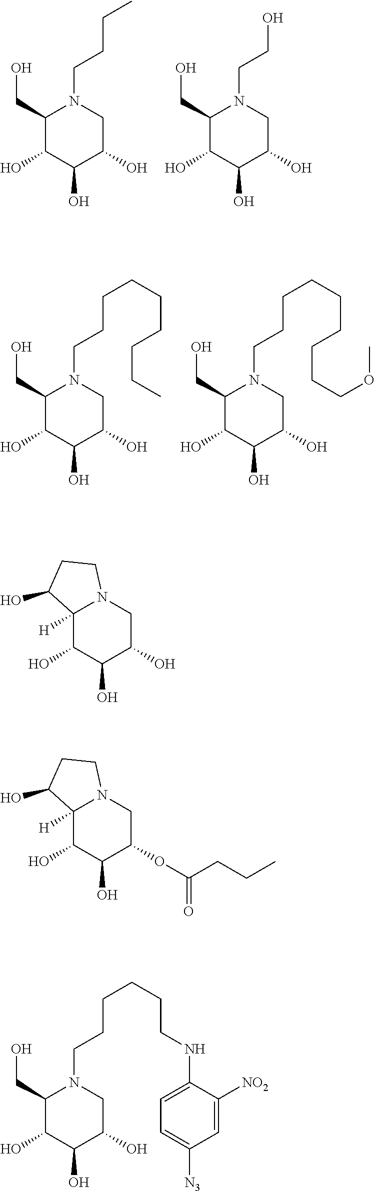 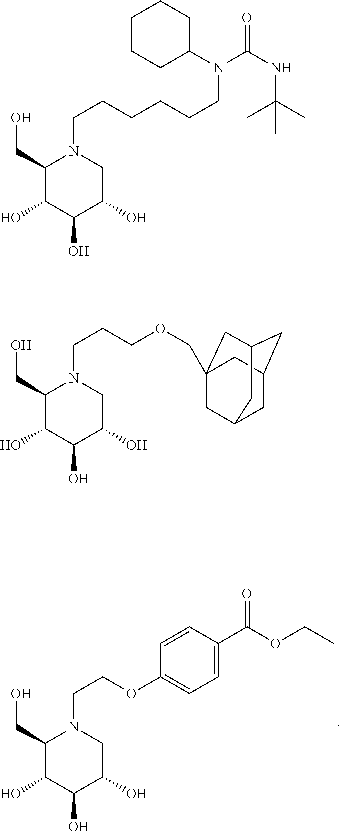 [SMILES: CC(C)(C)NC(=O)N(CCCCCCN1C[C@H](O)[C@@H](O)[C@H](O)[C@H]1CO)C1CCCCC1 ; OC[C@@H]1[C@@H](O)[C@H](O)[C@@H](O)CN1CCCOCC12CC3CC(CC(C3)C1)C2 ; CCOC(=O)c1ccc(OCCN2C[C@H](O)[C@@H](O)[C@H](O)[C@H]2CO)cc1]
or a pharmaceutically acceptable salt thereof.

8. The method of claim 1, wherein the α-glucosidase inhibitor is selected from: 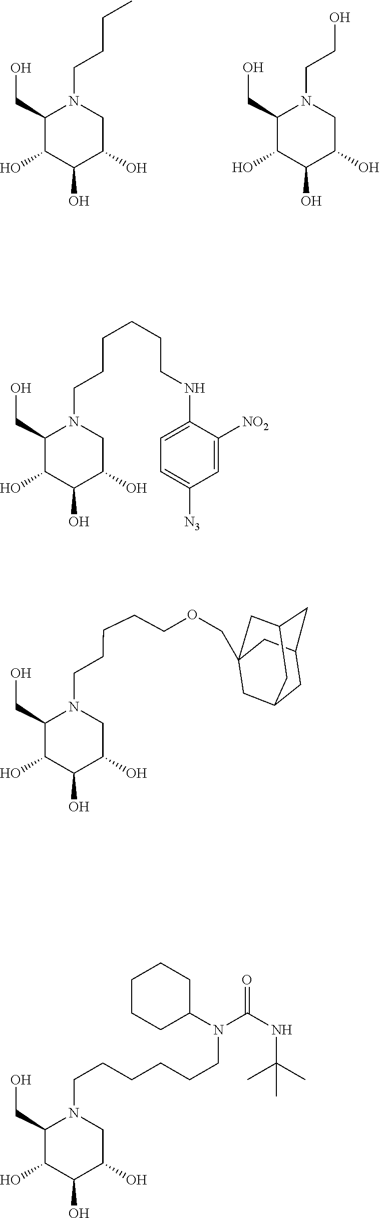 [SMILES: CCCCN1C[C@H](O)[C@@H](O)[C@H](O)[C@H]1CO ; OCCN1C[C@H](O)[C@@H](O)[C@H](O)[C@H]1CO ; [N-]=[N+]=Nc1ccc(NCCCCCCN2C[C@H](O)[C@@H](O)[C@H](O)[C@H]2CO)c([N+](=O)[O-])c1 ; OC[C@@H]1[C@@H](O)[C@H](O)[C@@H](O)CN1CCCCCOCC12CC3CC(CC(C3)C1)C2 ; CC(C)(C)NC(=O)N(CCCCCCN1C[C@H](O)[C@@H](O)[C@H](O)[C@H]1CO)C1CCCCC1]
or a pharmaceutically acceptable salt thereof.

9. The method of claim 1, wherein the α-glucosidase inhibitor is administered by oral inhalation.

10. The method of claim 1, wherein the α-glucosidase inhibitor is provided as a dry powder formulation.

11. The method of claim 10, wherein the dry powder formulation is a spray dried formulation.

12. The method of claim 10, wherein particles of the dry powder formulation have a mass median aerodynamic diameter of less than 10 μm.

13. The method of claim 1, wherein the dry powder formulation is administered in the form of an aerosol.

14. The method of claim 1, wherein the α-glucosidase inhibitor is administered in combination with one or more additional therapeutic agents.

15. The method of claim 14, wherein the additional therapeutic agent is selected from the group consisting of an anti-bacterial agent, an anti-viral agent, an anti-retroviral agent, an immunomodulator, an immunostimulant, an antibiotic and an anti-inflammatory agent.

16. (canceled)

17. (canceled)

18. An inhalable composition comprising an α-glucosidase inhibitor and a pharmaceutically acceptable carrier, diluent, adjuvant or excipient, wherein the α-glucosidase inhibitor is a compound of Formula (ID): 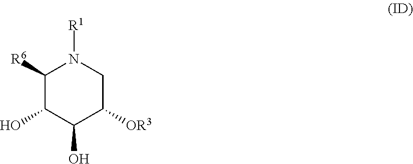
or a pharmaceutically acceptable salt thereof,
wherein:
R¹ is C₁₋₁₀alkyl optionally substituted with one or more R^{X} groups, or LR⁹;
L is a divalent linker group selected from C₁₋₁₀alkyl-O—, or C₁₋₁₀alkyl-NR⁷—;
R⁹ is selected from C₁₋₄alkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl, or C₁₋₉heteroaryl group optionally substituted with one or more R^{X} groups, R⁸, C₃₋₁₀cycloalkyl optionally substituted with one or more R^{X} groups, C₅₋₁₀cycloalkenyl optionally substituted with one or more R^{X} groups, C₂₋₆heterocycloalkyl optionally substituted with one or more R^{X} groups, C₆₋₁₀aryl optionally substituted with one or more R^{X} groups, or C₁-9heteroaryl optionally substituted with one or more R^{X} groups;
R³ is H or C(O)C₁₋₆alkyl;
R⁶ is CH₂—OH;
R⁷ and R⁸ are independently selected from H, C₁₋₄alkyl, C₃₋₆cycloalkyl, or C(O)NH-C₁₋₄alkyl;
or wherein R¹ and R⁶ together with the atoms to which they are attached form a 5-membered heterocycloalkyl ring substituted with a hydroxyl group; and
each R^{X} is independently selected from hydroxy, halo, nitro, azido, C₃₋₁₀cycloalkyl, C₁₋₄alkoxy and CO(O)C₁₋₄alkyl.

19. A method for treating or preventing a viral respiratory tract infection in a subject comprising pulmonary administration of a therapeutically effective amount of an α-glucosidase inhibitor to the subject, wherein the α-glucosidase inhibitor is: 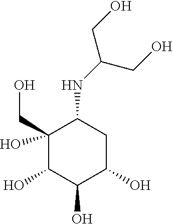 [SMILES: OCC(CO)N[C@@H]1C[C@H](O)[C@@H](O)[C@H](O)[C@@]1(O)CO]
or a pharmaceutically acceptable salt thereof.

20. (canceled)

21. (canceled)

22. An inhalable composition comprising an α-glucosidase inhibitor and a pharmaceutically acceptable carrier, diluent, adjuvant or excipient, wherein the α-glucosidase inhibitor is: 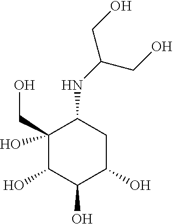 [SMILES: OCC(CO)N[C@@H]1C[C@H](O)[C@@H](O)[C@H](O)[C@@]1(O)CO]
or a pharmaceutically acceptable salt thereof.

23. (canceled)
